SEQUENCE LISTING (29 sequences)

SEQ ID NO: 1 - DNA, 5252 bp, Artificial Sequence
  Description of Artificial Sequence Synthetic polynucleotide
tagtttcgtaatatctatagcatcctcaaaaaatatattcgcatatattcccaagtcttcagttctatcttctaaaaaatcttcaacgtatggaatataataatctattttacctcttctgatatcattaatgatatagtttttgacactatcttctgtcaattgattcttattcactatatctaagaaacggatagcgtccctaggacgaactactgccattaatatctctattatagcttctggacataattcatctattataccagaattaatgggaactattccgtatctatctaacatagttttaagaaagtcagaatctaagacctgatgttcatatattggttcatacatgaaatgatctctattgatgatagtgactatttcattctctgaaaattggtaactcattctatatatgctttccttgttgatgaaggatagaatatactcaatagaatttgtaccaacaaactgttctcttatgaatcgtatatcatcatctgaaataatcatgtaaggcatacatttaacaattagagacttgtctcctgttatcaatatactattcttgtgataatttatgtgtgaggcaaatttgtccacgttctttaattttgttatagtagatatcaaatccaatggagctacagttcttggcttaaacagatatagtttttctggaacgaattctacaacattattataaaggactttgggtagataagtgggatgaaatcctattttaattaatgcgatagccttgtcctcgtgcagatatccaaacgcttttgtgatagtatggcattcattgtctagaaacgctctacgaatatctgtgacagatatcatctttagagaatatactagtcgcgttaatagtactacaatttgtattttttaatctatctcaataaaaaaattaatatgtatgattcaatgtataactaaactactaactgttattgggatctgaagcttaattcgccctttatggactgccgactccttcggtggtgccttgggattgttgagtgccggatgtgccgccggattccaaatcctgcagtgggactggttcggcggaccggccgagacaggtggacaagattctctcggacaggctgtgaatctgcttctgccgggcgcgccatctccgcctcctgttttttctggcttgccttgttcctcttggctcaggatacggatcggactggtacaaaatcttaataatccgaacagcgcgcagcaaggcctcatcggaatcgccggacctaccagccatctgctttgatataagattttgatgatcctcactgctttgaggagctcttcgtcgctgtctccgcttcttcctgccataggaaatgcctaagccttttttctgaaagcaaactagacaatgggcgctacagtgtttacaagcacagttattgcaagcagttttaggctgacttcctggatggttccagggctctaggttaggatctactggctccatggtggaagggcgaattaagctctagccctcgagggctagagtcactgttctttatgattctacttccttaccgtgcaataaattagaatatattttctacttttacgagaaattaattattgtatttattatttatgggtgaaaaacttactataaaaagtgggtgggtttggaattagtgatcagtttatgtatatcgcaactaccgggcatatggctatcgacatcgagaacattacccacatgataagagattgtatcagtttcgtagtcttgagtattggtattactatatagtatatagatgtcgatcgacggatcatcaagctggcggtacccaattcgcccttttatagcactttcctgatcccattacttactaatttatctacttgttcatttcctccaattcctttatgtgctggtacccatgacagatagaccctttccttttttattaattgttctattatttgattaactaactctgattcactcttatctggttgtgcttgaatgatccctaatgcatactgtgaatctgttactatgtttacctctgatcctgaatcttgcaaagctagacaaattgcttgcaactcagttttctgatttgtggtttcagttagagaaacaattttctgccttcctctgtcagtaacataccctgcttttcctattttggtgtccctattagctgctccatctacatagaaagtttctactcctgctatgggatctttctccagctggtaccataattttactaggggaggggtattaacaaattcccactcaggaatccaggtggcttgccaatagtctgtccaccatgtctcccatgtttctttttggatgggtaatctaaatttaggagtctttccccatattactatgctttccatggctattttctgcacagcctctgttaactgttttacatcattagtgtgggcagtcctcatttttgcatacttccctgttttcagatttttgaatggttcttggtaaatttgatatgtccattggtcctgcccctgtttctgtatttccgctatcaagtcttttgatgggtcataatatactccatgtactggttcttttagaatttccctgttttctgccaattctaattctgcttcttcagttagtggtactatgtctgttagtgttttggcccccctaaggagtttacaaagttgccttactttaattcctgggtaaatctgacttgcccagtttaattttcccactaacttctgtatatcattgacagtccagctatccttttctggtagctgtataggctgtactgtccatttgtcaggatggagttcataccccatccaaagaaatggaggttctttctgatgtttcttgtctggtgtggtaaatccccaccttaacagatgttctcttaactcctctatttttgctctatgttgccctatttctaagtcagatcctacatacaagttgttcatatattgatagatgactatttctggattttgtgccctaaagggctctaagattcttgtcatgctacactggaatattgctggtgatcctttccatccctgtggaagcacattatattgatacctaattcctggtgtttcattgtttacactaggtatggtgaatgcagtatatttcctgaagtcttcatataaaggaactgaaaaatatgcatcccccacatccagtactgtcactgattttttcttttttaaccctgctgggtgtggtattcctaattgaacttcccaaaaatcttgagttcttttattgagttccctgaaatctactaattttctccacttagtactgtccttcttttttatggcaaatattggagtgttatatggattttcaggcccaatttttgtaatttttccttccttctccatttcatcacaaattgctgttaatgcttttattttctcttctgtcaatggccattgtttaacctttggcccatccattcctggctttaattttactggtacagtttcaatgggactgattggccccatgcagtctttgtaatactccggatgtagctcgcgggccatgtgtctgtgtgctaggtggctgtcaaacttccactgcaacacttctctgtgttcatcctccattccatgctggcacacagggtgtagcaaacagttgttttctccttcgttggcctcttctacttcccttgggtcaactggtactagcttgaagcaccacccaaaggttagtgggaatctgactcctggtcccggtgtgtagttctgccaatcagggaagaagccttgtgtgtgatagacccacaaatcaaggatctcttgccttttcttagagtaaattaacccttccagtccccccttttcttttaaaaagaagctgagatcgaatgctcccttaaaagtcattggtcttaaaggcacctgaggtctgactggaaagcctacggcggcggcggcggcttgcgctctcagccaggcacaatcagcattagtggtgtctgtgttgctgcttgtaagtgctccatatttatctaagtcttgagacgctgctcctactccttctgctgctggctcagttcgtctcattcgttctcttacagcaggccatccaactatgctgcattttgaccacttgccccccatggtggaagggcgaattgggctgcaggaatttcgacttcgagcttatttatattccaaaaaaaaaaaataaaatttcaatttttaagctcgagctcgaattcatcgatgattccctagaatcagaatctaatgatgacgtaaccaagaagtttatctactgccaatttagctgcattatttttagcatctcgtttagattttccatctgccttatcgaatactcttccgtcgatatctacacaggcataaaatgtaggagagttactaggccccactgattcaatacgaaaagaccaatctctcttagttatttggcagtactcattaataatggtgacagggttagcatctttccaatcaataatttttttagccggaataacatcatcaaaagacttatgatcctctctcattgatttttcgcgggatacatcatctattatggcgtcagccataacatcagcatccggcttatccgcctccgttgtcataaaccaacgaggaggaatatcgtcggagctgtacaccatagcactacgttgaagatcgtacagagctttattaacttctcgcttctccatattaagttgtctagttagttgtgcagcagtagctccttcgattccaatgtttttaatagccgcacacacaatctctgcgtcagaacgctcgtcaatatagatcttagacatttttagagagaactaacacaaccagcaataaaactaatttattttatcatttttttattcatcatcctctggtggttcgtcgtttctatcgaatgtggatctgattaacccgtcatctataggtgatgctggttctggagattctggaggagatggattattatctggaagaatctctgttatttccttgttttcatgtatcgattgcgttgtaacattaagattgcgaaatgctctaaatttgggaggcttaaagtgttgtttgcaatctctacacgcatgtctaactagtggaggttcgtcagcggctctagtttgaatcatcatcggcgtagtattcctacttttacagttaggacacggtgtattgtatttctcgtcgagaacgttaaaata

SEQ ID NO: 2 - DNA, 6815 bp, Artificial Sequence
  Description of Artificial Sequence Synthetic polynucleotide
aataaagcgagactatttgaacgagggttccatggcagattctgccgatttagtagtactaggtgcttactatggtaaaggagcaaagggtggtatcatggcagtctttctaatgggttgttacgacgatgaatccggtaaatggaagacggttaccaagtgttcaggacacgatgataatacgttaagggagttgcaagaccaattaaagatgattaaaattaacaaggatcccaaaaaaattccagagtggttagtagttaataaaatctatattcccgattttgtagtagaggatccaaaacaatctcagatatgggaaatttcaggagcagagtttacatcttccaagtcccataccgcaaatggaatatccattagatttcctagatttactaggataagagaggataaaacgtggaaagaatctactcatctaaacgatttagtaaacttgactaaatcttaatagttacatacaaattaaaataacactatttagttggtggtcgccatggatggtgttattgtatactgtctaaacgcgttagtaaaacatggcgaggaaataaatcatataaaaaatgatttcatgattaaaccatgttgtgaaaaagtcaagaacgttcacattggcggacaatctaaaaacaatacagtgattgcagatttgccatatatggataatgcggtatccgatgtatgcaattcactgtataaaaagaatgtatcaagaatatccagatttgctaatttgataaagatagatgacgatgacaagactcctactggtgtatataattattttaaacctaaagatgccattcctgttattatatccataggaaaggatagagatgtttgtgaactattaatctcatctgataaagcgtgtgcgtgtatagagttaaattcatataaagtagccattcttcccatggatgtttccttttttaccaaaggaaatgcatcattgattattctcctgtttgatttctctatcgatgcggcacctctcttaagaagtgtaaccgataataatgttattatatctagacaccagcgtctacatgacgagcttccgagttccaattggttcaagttttacataagtataaagtccgactattgttctatattatatatggttgttgatggatctgtgatgcatgcaatagctgataatagaacttacgcaaatattagcaaaaatatattagacaatactacaattaacgatgagtgtagatgctgttattttgaaccacagattaggattcttgatagagatgagatgctcaatggatcatcgtgtgatatgaacagacattgtattatgatgaatttacctgatgtaggcgaatttggatctagtatgttggggaaatatgaacctgacatgattaagattgctctttcggtggctggagcttaaaaattgaaattttattttttttttttggaatataaataagctcgaagtcgaaattcctgcagcccggccgccagtgtgatggatatctgcagaattcgcccttccaccatgagagtgagggggacactgaggaattatcaacaatggtggatatggggcgtcttaggcttttggatgttaatgatttgtaatgtgggaggaaacttgtgggtcacagtctattatggggtacctgtgtggaaagaagcaaaaactactctattctgtgcatcagatgctaaagcacatgagagagaggtgcataatgtctgggctacacatgcctgtgtacccacagaccccaacccacaagaaatggttttggaaaatgtaacagaaaattttaacatgtggaaaaatgacatggtgaatcagatgcatgaggatgtaatcagtttatgggatcaaagcctaaagccatgtgtaaagttgaccccactctgtgtccctttaaaatgtaaaaatgttacctacaatgagagtatgcaggaaataaaaaattgctctttcaatgcaaccacagatttaagagataggaagcagacagtgcaggcactcttttataaacttgatatagtatcacttaatgagaagaactctagtgagtattatagattaataaattgtaatacctcagccataacacaagcctgtccaaaggtcacttttgatccaatccctatacattattgtactccggctggttatgcgattctaaagtgtaatgagcagacattcaatgggacaggaccatgccataatgttagcacagtacaatgtacacatggaattaagccagtagtatcaactcaactactgttaaatggtagcctagcagaaagagagataataattagatctgaaaatttgacaaacaatgtcaaaacaataatagtacatcttaatcaatctgtagaaattgtgtgtacaagacccaacaataatacaagaaaaagtataaggataggaccaggacaaacattctatgcaacaggagacataatgggagacataagacaagcacattgtaacattagtgcaggaaaatggaatgaaactttacaaagggtaggtaacaaattagcagaacacttccctaataaaacaataaaatttgcaccatcttcaggaggggacctagaaattacaacacatagctttaattgtagaggagaattcttctattgtaatacatcaggcctgtttaatggtacatacaattggacagaaagtaattcaagctcaatcatcacaatcccatgcagaataaagcaaattataaacatgtggcaggaggtaggacgagcaatgtatgcccctcccattgaaggaaacataacatgcaaatcaaatatcacaggactactattggtacgtgatggaggaacagaggcaaatacgacagagacattcagacctggaggaggagatatgaggaacaattggagaagtgaattatataaatataaagtggtagaaattaagccattgggagtagtacccacagaagcaaaaaggagagtggtggagagagaaaaaagagcagtgggaataggagctgtgttccttgggttcttgggagcagcaggaagcactatgggcgcggcgtcaataacgctgacggcacaggccagacaattgttgtctggtatagtgcaacagcaaagcaatttgctgagggctatagaagcgcaacagcatctgttgcagctcacggtctggggcattaagcagctccagacaagagtcctggctatagaaagatacctaaaggatcaacagctcctagggatttggggctgctctggaaaactcatctgcactactgctgtaccttggaactccagttggagtaacagatctcaagaagagatttggaataacatgacctggatgcagtgggatagagaaattagtaattacacaaacacaatatacaggttgcttgaagactcgcaaaaccagcaggaaaaaaatgaaaaggatttattagcattggacagttggaaaaatctatggagttggtttgacataacaaattggctgtggtatataaaaatattcataatgatagtaggaggcttgataggtttaagaataatttttgctgtgctctctatagtgaatagagttaggcagggatactcacctttgtcgtttcagacccttaccccgaacccagggggacacgacaggctcggaagaatcgaagaagaaggtggagagcaagacaaaaacagatccattcgattagtgaacggattcttagcacttgcctgggacgatctgcggaacctgtgcctcttcagctaccaccgattgagagacttcatattagtgatagcgagagtggtggaacttctgggacgcaacagtctcaggggactacagaagggatgggaaggccttaaatatctgggaagtcttgtgcagtattggggtctggagctaaaaaagagtgctattagtctgtttgatatcatagcaatagcagtagctgaaggaacagatagaattatagaattagtacaaggaatttgtagagctatccgcaacatacctagaagaataagacagggctttgaagcagctttgcaataaaagggcgaattccagcacactggcggccgttactaggggtaccgccgggagatctgggtaaagttacaaacaactaggaaattggtttatgatgtataatttttttagtttttatagattctttattctatacttaaaaaatgaaaataaatacaaaggttcttgagggttgtgttaaattgaaagcgagaaataatcataaattatttcattatcgacagtttacccacacggcggatcgactctagctcgaattcgcccttccaccatgggtgcgagagcgtcaatattaagaggggaaaaattagataaatgggaaaaaattaggttaaggccagggggaaagaaacactatatgctaaaacacctagtatgggcaagcagggagctggacagattcgcgcttaaccctggccttttagagacatcagaaggctgtaaacaaataataaaacagctacaaccagctcttcagacaggaacagaagaacttagatcattacacaacacagtagtaactctctattgtgtacatgcagggatagaagtacgagacaccaaagaagccttagacaagatagaggaagaacaaaacaaaaatcagcaaaaaacacagcaggcaaaagaggctgacgagaaggtcagtcaaaattatcctatagtgcagaatctccaagggcaaatggtacaccaggccctatcacctagaactttgaatgcatgggtaaaagtaatagaggagaaggcttttagcccagaggtaatacccatgtttacagcattatcagaaggagccaccccacaagatttaaataccatgttaaatacagtggggggacatcaagcagccatgcaaatgttaaaagatactatcaatgaagaggctgcagaatgggatagattacatccaatacatgcagggcctattgcaccaggccaaatgagagaaccaaggggaagtgacatagcaggaactactagtagccttcaggaacaaatagcatggatgacgggtaacccacctgttccagtgggagacatctataaaagatggataattctggggttaaataaaatagtaagaatgtatagccctgttagcattttggacataaaacaagggccaaaggaaccctttagagactatgtagaccggttctttaaaactctaagagctgaacaagctacacaagatgtaaaaaattggatgacagacaccttgttggtccaaaatgcgaatccagattgtaagaccattttaagagcattaggaccaggggcttcactagaagagatgatgacagcatgtcagggagtgggaggacctagccacaaagcaagagtgttggctgaggcaatgagccaaacaaacagtgccatactgatgcagaaaagcaattttaaaggctctaaaagaattattaaatgtttcaactgtggcaaggaagggcacctagccagaaattgcagggcccctaggaaaaaaggctgttggaaatgtggaaaggaaggacaccaaatgaaagactgtactgagaggcaggctaattttttagggaaaatttggccttcccacaaggggaggccagggaatttcctccagagcagaccagagccgacagccccaccagcagagagcttcaggttcgaggagacacccccagctccaaagcaggagccgaaagacagggaacccttaacttccctcagatcactctttggcagcgaccccttgtctcaataaaaagggcgaatttcgagggaaagttttataggtagttgatagaacaaaatacataattttgtaaaaataaatcactttttatactaatatgacacgattaccaatacttttgttactaatatcattagtatacgctacaccttttcctcagacatctaaaaaaataggtgatgatgcaactttatcatgtaatcgaaataatacaaatgactacgttgttatgagtgcttggtataaggagcccaattccattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcgcctacaaatgacactgataaagtagattatgaagaatactccacagagttgattgtaaatacagatagtgaatcgactatagacataatactatctggatctacacattcaccggaaactagttctgagaaacctgattatatagataattctaattgctcgtcggtattcgaaatcgcgactccggaaccaattactgataatgtagaagatcatacagacaccgtcacatacactagtgatagcattaatacagtaagtgcatcatctggagaatccacaacagacgagactccggaaccaattactgataaagaagaagatcatacagttacagacactgtctcatacactacagtaagtacatcatctggaattgtcactactaaatcaaccaccgatgatgcggatctttatgatacgtacaatgataatgatacagtaccatcaactactgtaggcggtagtacaacctctattagcaattataaaaccaaggactttgtagaaatatttggtattaccgcattaattatattgtcggccgtggcaatattctgtattacatattatatatataa

SEQ ID NO: 3 - protein, 207 aa, Human immunodeficiency virus type 1
MGGKWSKCSIVGWPAVRERMRRTEPAAEGVGAASQDLDKYGALTSSNTDTTNADCAWLRAQEEEEEVGFPVRPQVPLRPMTFKGAFDLSFFLKEKGGLEGLIYSKKRQEILDLWVYHTQGFFPDWQNYTPGPGVRFPLTFGWCFKLVPVDPREVEEANEGENNCLLHPVCQHGMEDEHREVLQWKFDSHLAHRHMARELHPEYYKDC

SEQ ID NO: 4 - DNA, 624 bp, Human immunodeficiency virus type 1
atggggggcaagtggtcaaaatgcagcatagttggatggcctgctgtaagagaacgaatgagacgaactgagccagcagcagaaggagtaggagcagcgtctcaagacttagataaatatggagcacttacaagcagcaacacagacaccactaatgctgattgtgcctggctgagagcgcaagaggaggaagaagaagtaggctttccagtcagacctcaggtgcctttaagaccaatgacttttaagggagcattcgatctcagcttctttttaaaagaaaaggggggactggaagggttaatttactctaagaaaaggcaagagatccttgatttgtgggtctatcacacacaaggcttcttccctgattggcagaactacacaccgggaccaggagtcagattcccactaacctttgggtggtgcttcaagctagtaccagttgacccaagggaagtagaagaggccaacgaaggagaaaacaactgtttgctacaccctgtgtgccagcatggaatggaggatgaacacagagaagtgttgcagtggaagtttgacagccacctagcacacagacacatggcccgcgagctacatccggagtattacaaagactgctga

SEQ ID NO: 5 - protein, 107 aa, Human immunodeficiency virus type 1
MAGRSGDSDEALLRAVRIIKILYQSNPYPEPRGTRQARKNRRRRWRARQKQIHSLSERILSTCLGRSAEPVPLQLPPLERLHISGSESGGTSGTQQSQGTTEGVGSP

SEQ ID NO: 6 - DNA, 2735 bp, Human immunodeficiency virus type 1
atggcaggaagaagcggagacagcgacgaagcgctcctccgagcagtgaggatcatcaaaatcttatatcaaagcagtaagtatctgtaataatagatttagattatagattaggagtaggagcattgatagtagcatttatcatagcaatagtagtgtggaccatagtatatataaaatataggaaattgttaagacaaagaagaatagactggttaattaaaagaattagggaaagagcagaagacagtggcaatgagagtgagggggatactgaggaattatcaacaatggtggatatggggcatcttaggcttttggatgttaatgatatgtaatgtggtaggaaatttgtgggtcacagtctattatggggtacctgtgtggaaagaagcaaaaactactttattctgtgcatcagatgctaaagcatatgagaaagaagtgcataatgtctgggctacacatgcctgtgtacctacagaccccaacccacaagaaatggttttggaaaatgtaacagaaaattttaacatgtggaaaaatgacatggtgaatcagatgcatgaggatgtaatcagcttatgggatcaaagcctaaagccatgtgtaaagttgaccccactctgtgtcactttagaatgtagtgagtataatggtaccagtaaggctaatgctaccaataatgttaatgctaccagtaatggtaatgctactagtaatggggaagaaatacaacagtgttttttcaatgtaaccacagaaatgagagataagaagcagagggtgcatgcacttttttatagacttgatctagtaccacttgataatgagaataagagcagctttagcaactctagtaagacttatagattaataaattgtaatacctcagccataacacaagcctgtccaaaggtcacttttgatccaattcctatacattattgtactccagctggttatgcgattctaaagtgtaatgagaagacattcaatgggacaggactatgccagaatgtcagcacagtacaatgtacacatggaattaagccagtggtatcaactcaactactgttaaatggtagcctagcagaaggagagataataattagatctgaaaatctgacagacaatgtcaaaacaataatagtacatcttaatcaatctgtagaaattgagtgtgtaagacccaacaataatacaagagaaagtataaggataggaccaggacaaacattctatgcaacaggagaaataataggagacataagacaagcacattgtaacattagtgctgacagatggaatgaaactttacaatgggtaggtgaaaagttagcagaacacttccctaataaaacaataaaatttgcaccatcctcaggaggggacctagaaattacaacacatagctttaattgtagaggggaatttttctattgcaatacatcaagcctgtttgatagcctgtttaatcctaatggtacaagaaatgatacaaacttaaccattacaatcccatgcagaataaaacaaattataaacatgtggcaggaggtaggacgagcaatgtatgcccctcccattgcaggaaacataacatgtaaatcaaacatcacaggactactattggtgcgtgatggaggaagaggtaatgatacagagaataatacagagatattcagacctggaggaggagatatgaggaacaattggagaagtgaattatataaatataaagtggtagaaattaagccattgggagtagcacccactaaagcaaaaaggagagtggtggagagagaaaaaagagcagtagtgggactaggagctgtgttccttgggttcttgggagcagcaggaagcactatgggcgcggcgtcaataacgctgacggtacaggccagacaattgttgtctggtatagtgcaacagcaaagcaatttgctgagggctatagaggcgcaacagcatctgttgcaactcacggtctggggcattaagcagctccagacaagagtcctggctatagaaagatacctaaaggatcaacagctcctagggatttggggctgctctggaaaactcatctgcaccactgctgtaccttggaactccagttggagtaacaaatctcaaatagaaatttgggagaacatgacctggatgcagtgggacagagaaattaataattacacacaaacaatatataggttgcttgaggactcgcaaaaccagcaggaaagaaatgaaaaggatttattagcattggacagttgggaaagtctgtggaattggtttagcatatcaaagtggctgtggtatataaaaatattcataatgatagtaggaggcttgataggtttaagaataatttttgctgtgctttctatagtgaatagagttaggcagggatactcacctttgtcatttcagacccttaccccgaacccagggggacccgacaggctcggaagaatcgaagaagaaggtggagagcaagacaaaaacagatccattcgcttagtgaacggattcttagcacttgcctgggacgatctgcggagcctgtgcctcttcagctaccaccgcttgagagacttcatattagtggcagtgagagcggtggaacttctgggacgcagcagtctcaggggactacagagggggtgggaagcccttaaatatctgggaagtcttgtgcagtattggggtatagagctaaaaaggag

SEQ ID NO: 7 - protein, 491 aa, Human immunodeficiency virus type 1
MGARASILRGEKLDKWEKIRLRPGGKKHYMLKHLVWASRELDRFALNPGLLETSEGCKQIIKQLQPALQTGTEELRSLHNTVVTLYCVHAGIEVRDTKEALDKIEEEQNKNQQKTQQAKEADEKVSQNYPIVQNLQGQMVHQALSPRTLNAWVKVIEEKAFSPEVIPMFTALSEGATPQDLNTMLNTVGGHQAAMQMLKDTINEEAAEWDRLHPIHAGPIAPGQMREPRGSDIAGTTSSLQEQIAWMTGNPPVPVGDIYKRWIILGLNKIVRMYSPVSILDIKQGPKEPFRDYVDRFFKTLRAEQATQDVKNWMTDTLLVQNANPDCKTILRALGPGASLEEMMTACQGVGGPSHKARVLAEAMSQTNSAILMQKSNFKGSKRIIKCFNCGKEGHLARNCRAPRKKGCWKCGKEGHQMKDCTERQANFLGKIWPSHKGRPGNFLQSRPEPTAPPAESFRFEETPPAPKQEPKDREPLTSLRSLFGSDPLSQ

SEQ ID NO: 8 - DNA, 1476 bp, Human immunodeficiency virus type 1
atgggtgcgagagcgtcaatattaagaggggaaaaattagataaatgggaaaaaattaggttaaggccagggggaaagaaacactatatgctaaaacacctagtatgggcaagcagggagctggacagattcgcgcttaaccctggccttttagagacatcagaaggctgtaaacaaataataaaacagctacaaccagctcttcagacaggaacagaagaacttagatcattacacaacacagtagtaactctctattgtgtacatgcagggatagaagtacgagacaccaaagaagccttagacaagatagaggaagaacaaaacaaaaatcagcaaaaaacacagcaggcaaaagaggctgacgagaaggtcagtcaaaattatcctatagtgcagaatctccaagggcaaatggtacaccaggccctatcacctagaactttgaatgcatgggtaaaagtaatagaggagaaggcttttagcccagaggtaatacccatgtttacagcattatcagaaggagccaccccacaagatttaaataccatgttaaatacagtggggggacatcaagcagccatgcaaatgttaaaagatactatcaatgaagaggctgcagaatgggatagattacatccaatacatgcagggcctattgcaccaggccaaatgagagaaccaaggggaagtgacatagcaggaactactagtagccttcaggaacaaatagcatggatgacgggtaacccacctgttccagtgggagacatctataaaagatggataattctggggttaaataaaatagtaagaatgtatagccctgttagcattttggacataaaacaagggccaaaggaaccctttagagactatgtagaccggttctttaaaactctaagagctgaacaagctacacaagatgtaaaaaattggatgacagacaccttgttggtccaaaatgcgaatccagattgtaagaccattttaagagcattaggaccaggggcttcactagaagagatgatgacagcatgtcagggagtgggaggacctagccacaaagcaagagtgttggctgaggcaatgagccaaacaaacagtgccatactgatgcagaaaagcaattttaaaggctctaaaagaattattaaatgtttcaactgtggcaaggaagggcacctagccagaaattgcagggcccctaggaaaaaaggctgttggaaatgtggaaaggaaggacaccaaatgaaagactgtactgagaggcaggctaattttttagggaaaatttggccttcccacaaggggaggccagggaatttcctccagagcagaccagagccgacagccccaccagcagagagcttcaggttcgaggagacacccccagctccaaagcaggagccgaaagacagggaacccttaacttccctcagatcactctttggcagcgaccccttgtctcaataa

SEQ ID NO: 9 - protein, 101 aa, Human immunodeficiency virus type 1
MEPVDPNLEPWNHPGSQPKTACNNCYCKHCSYHCLVCFQKKGLGISYGRKKRRQRRRAPQSSEDHQNLISKQPLPRTQGDPTGSEESKKKVESKAKTDPFA

SEQ ID NO: 10 - DNA, 2621 bp, Human immunodeficiency virus type 1
atggagccagtagatcctaacctagagccctggaaccatccaggaagtcagcctaaaactgcttgcaataactgttattgtaaacactgtagctaccattgtctagtttgctttcagaaaaaaggcttaggcatttcctatggcaggaagaagcggagacagcgacgaagagctcctcaaagcagtgaggatcatcaaaatcttatatcaaagcagtaagtatctgtaatgttagatttagattataaattagcagtaggagcattgatagtagcactaatcatagcaatagttgtatggatcatagcatatatagaatataggaaattggtaaaacaaaggaaaatagactggttaattaaaagaattagggagagagcagaagacagtggcaatgagagtgagggggacactgaggaattatcaacaatggtggatatggggcgtcttaggcttttggatgttaatgatttgtaatgtggggggaaacttgtgggtcacagtctattatggggtacctgtgtggaaagaagcaaaaactactctattctgtgcatcagatgctaaagcatatgagaaagaagtgcataatgtctgggctacacatgcctgtgtacccacagaccccaacccacaagaaatacctttgggaaatgtaacagaaaattttaacatgtggaaaaatgacatggtgaatcagatgcatgaggatgtaatcagtttatgggatcaaagcctaaagccatgtgtaaagttgaccccactctgtgtcactttagaatgtaaaaatgttaaaaatgatagtacccacaatgagacctacactgagagcgtgaaggaaataaaaaattgctctttcaatgcaaccacagaaataagagataggaagcagacagtgtatgcactcttttatagacttgatatagtacaacttaactctgatgataagaaaaactctagtgagtattatagattaataaattgtaatacctcagccataacacaagcctgtccaaaggtcacttttgatccaattcctatacactattgtactccggctggttatgcgattctaaagtgtaaggataagacattcaatgggacaggaccatgccataatgttagcacagtacaatgtacacatggaattaagccagtagtatcaacgcaactactgttaaatggtagcctagcagaaggagagataataattagatctgaaaatctgacaaacaatgccaaaacaataatagtacatcttaatcaatctgtacaaattgtgtgtacaagacccaacaataatacaagaaaaagtataaggataggaccaggacaaacattctatgcaacaggagaaataataggagacataagacaagcacattgtaacattagtaaagaaaattggactgacacgttacaaagggtaagtaaaaaattagcagaacacttccctaataaaacaataaaatttgattcaccctcaggaggggacctagaaattacaacacatagctttaattgtagaggagaatttttctattgtaatacatcaggcctgtttaatggtacatacaatacatcatcagatggtaattcaagttcaaccatcacaatcccatgcagaataaagcaaattataaacatgtggcaggaggtaggacgagcaatgtatgcccctcccattgaaggaaacataacatgtaaatcaaatatcacaggactactattggtacgtgatggaggagcagaggcaaagacaaataatacagagacattcagacctggaggaggagatatgagggacaattggagaagtgaattatataaatataaagtggtagaaattaagccattaggagtagcacccactacagcaaaaaggagagtggtggagagagaaaaaagagcagtgggaataggagctttgttccttgggttcttgggagcagcaggaagcactatgggcgcggcgtcaataacgctgacggtacaggccagacaattgttgtctggtatagtgcaacagcaaagcaatttgctgagggctatagaggcgcaacagcatctgttgcaactcacggtctggggcattaagcagctccagacaagagtcctggctatagaaagatacctaaaggatcaacagctcctagggatttggggctgctctggaaaactcatctgcactactgctgtaccttggaactccagttggagtaacagatctcaacaagatatttgggataacatgacctggatgcagtgggatagagaaattagtaattacacaaacacaatatacaggttgcttgaagactcgcaaaaccagcaggaaaaaaatgaaaaagatttattagcattggacagttggaaaaatctatggagttggtttgacataacaaattggctgtggtatataaaaatcttcataatgatagtaggaggcttgataggtttaagaataatttttgctgtgctctctatagtgaatagagttaggcagggatactcacctttgtcgtttcagacccctaccccgaacccagggggacccgacaggctcggaagaatcgaagaagaaggtggagagcaaggcaaagacagatccattcgcttag

SEQ ID NO: 11 - protein, 999 aa, Human immunodeficiency virus type 1
FFRENLAFPQGEAREFPPEQTRANSPTSRELQVRGDNPSSKAGAERQGTLNFPQITLWQRPLVSIRVGGQIKEALLDTGADDTVLEEVNLPGKWKPKMIGGIGGFIKVRQYDQIPIEICGKKAIGTVLVGPTPVNIIGRNMLTQLGCTLNFPISPIETVPVKLKPGMDGPKVKQWPLTEEKIKALTAICDEMEKEGKITKIGPENPYNTPIFAIKKKDSTKWRKLVDFRELNKRTQDFWEVQLGIPHPAGLKKKKSVTVLDVGDAYFSVPLYEDFRKYTAFTIPSVNNETPGIRYQYNVLPQGWKGSPAIFQCSMTRILEPFRAQNPEIVIYQYMDDLYVGSDLEIGQHRAKIEELREHLLRWGFTTPDKKHQKEPPFLWMGYELHPDKWTVQPIQLPEKDSWTVNDIQKLVGKLNWASQIYPGIKVRQLCKLLRGAKTLTDIVPLTEEAELELAENREILKEPVHGVYYDPSKDLIAEIQKQGQDQWTYQIYQEPFKNLKTGKYAKMRTAHTNDVKQLTEAVQKIAMESIVIWGKTPKFRLPIQKETWETWWTDYWQATWIPEWEFVNTPPLVKLWYQLEKDPIAGVETFYVDGAANRDTKIGKAGYVTDRGRQKIVSLTETTNQKTELQAICLALQDSGSEVNIVTDSQYALGIIQAQPDKSESELVNQIIEQLIKKERVYLSWVPAHKGIGGNEQVDKLVSNGIRKVLFLDGIDKAQEEHEKYHSNWRAMASDFNLPPVVAKEIVASCDQCQLKGEAMHGQVDCSPGIWQLDCTHLEGKIILVAVHVASGYIEAEVIPAETGQETAYFILKLAGRWPVKVIHTDNGSNFTSAAVKAACWWAGIQQEFGIPYNPQSQGVVESMNKELKKIIRQVRDQAEHLKTAVQMAVFIHNFKRKGGIGGYSAGERIIDIIATDIQTKELQKQITKIQNFRVYYRDSRDPIWKGPAKLLWKGEGAVVIQDNSDIKVVPRRKAKIIKDYGKQMAGADCVAGRQDED

SEQ ID NO: 12 - DNA, 3000 bp, Human immunodeficiency virus type 1
ttttttagggaaaatttggccttcccacaaggggaggccagggaatttcctccagagcagaccagagccaacagccccaccagcagagagcttcaggttcgaggagacaaccccagctccaaagcaggagccgaaagacagggaacccttaacttccctcagatcactctttggcagcgaccccttgtctcaataagagtagggggccagataaaagaggctctcttagacacaggagcagatgatacagtattagaagaagtaaatttgccaggaaaatggaaaccaaaaatgataggaggaattggaggttttatcaaagtaagacaatatgatcaaatacctatagaaatttgtggaaaaaaggctataggtacagtattagtaggacccacacctgtcaacataattggaaggaatatgttgactcagcttggatgcacactaaattttccaatcagtcccattgaaactgtaccagtaaaattaaagccaggaatggatgggccaaaggttaaacaatggccattgacagaagagaaaataaaagcattaacagcaatttgtgatgaaatggagaaggaaggaaaaattacaaaaattgggcctgaaaatccatataacactccaatatttgccataaaaaagaaggacagtactaagtggagaaaattagtagatttcagggaactcaataaaagaactcaagatttttgggaagttcaattaggaataccacacccagcagggttaaaaaagaaaaaatcagtgacagtactggatgtgggggatgcatatttttcagttcctttatatgaagacttcaggaaatatactgcattcaccatacctagtgtaaacaatgaaacaccaggaattaggtatcaatataatgtgcttccacagggatggaaaggatcaccagcaatattccagtgtagcatgacaagaatcttagagccctttagggcacaaaatccagaaatagtcatctatcaatatatggatgacttgtatgtaggatctgacttagaaatagggcaacatagagcaaaaatagaggagttaagagaacatctgttaaggtggggatttaccacaccagacaagaaacatcagaaagaacctccatttctttggatggggtatgaactccatcctgacaaatggacagtacagcctatacagctaccagaaaaggatagctggactgtcaatgatatacagaagttagtgggaaaattaaactgggcaagtcagatttacccaggaattaaagtaaggcaactttgtaaactccttaggggggccaaaacactaacagacatagtaccactaactgaagaagcagaattagaattggcagaaaacagggaaattctaaaagaaccagtacatggagtatattatgacccatcaaaagacttgatagcggaaatacagaaacaggggcaggaccaatggacatatcaaatttaccaagaaccattcaaaaatctgaaaacagggaagtatgcaaaaatgaggactgcccacactaatgatgtaaaacagttaacagaggctgtgcagaaaatagccatggaaagcatagtaatatggggaaagactcctaaatttagattacccatccaaaaagaaacatgggagacatggtggacagactattggcaagccacctggattcctgagtgggaatttgttaatacccctcccctagtaaaattatggtaccagctggagaaagatcccatagcaggagtagaaactttctatgtagatggagcagctaatagggacaccaaaataggaaaagcagggtatgttactgacagaggaaggcagaaaattgtttctctaactgaaaccacaaatcagaaaactgagttgcaagcaatttgtctagctttgcaagattcaggatcagaggtaaacatagtaacagattcacagtatgcattagggatcattcaagcacaaccagataagagtgaatcagagttagttaatcaaataatagaacaattaataaaaaaggaaagggtctatctgtcatgggtaccagcacataaaggaattggaggaaatgaacaagtagataaattagtaagtaatgggatcaggaaagtgctatttctagatggaatagataaagctcaagaagagcatgaaaagtatcacagcaattggagagcaatggctagtgactttaatctgccacccgtagtagcaaaagaaatagtagccagctgtgatcaatgtcagctaaaaggggaagccatgcatggacaagtagactgtagtccagggatatggcaattagattgtacacatttagaaggaaaaatcatcctggtagcagtccatgtagccagtggctacatagaagcagaggttatcccagcagaaacaggacaagaaacagcatactttatactaaaattagcaggaagatggccagtcaaagtaatacatacagacaatggtagtaatttcaccagtgctgcagttaaggcagcctgttggtgggcaggtatccaacaggaatttggaattccctacaatccccaaagtcagggagtagtagaatccatgaataaagaattaaagaaaattataaggcaggtaagagatcaagctgagcaccttaagacagcagtacaaatggcagtatttattcacaattttaaaagaaaaggggggattggggggtacagtgcaggggaaagaataatagacataatagcaacagacatacaaactaaagaattacaaaaacaaattacaaaaattcaaaattttcgggtttattacagagacagcagagaccccatttggaaaggaccagccaaactactctggaaaggtgaaggggcagtagtaatacaagataatagtgacataaaggtagtaccaaggaggaaagcaaaaatcattaaggactatggaaaacagatggcaggtgctgattgtgtggcaggtagacaggatgaagattag

SEQ ID NO: 13 - protein, 843 aa, Human immunodeficiency virus type 1
MRVRGTLRNYQQWWIWGVLGFWMLMICNVGGNLWVTVYYGVPVWKEAKTTLFCASDAKAHEREVHNVWATHACVPTDPNPQEMVLENVTENFNMWKNDMVNQMHEDVISLWDQSLKPCVKLTPLCVPLKCKNVTYNESMQEIKNCSFNATTDLRDRKQTVQALFYKLDIVSLNEKNSSEYYRLINCNTSAITQACPKVTFDPIPIHYCTPAGYAILKCNEQTFNGTGPCHNVSTVQCTHGIKPVVSTQLLLNGSLAEREIIIRSENLTNNVKTIIVHLNQSVEIVCTRPNNNTRKSIRIGPGQTFYATGDIMGDIRQAHCNISAGKWNETLQRVGNKLAEHFPNKTIKFAPSSGGDLEITTHSFNCRGEFFYCNTSGLFNGTYNWTESNSSSIITIPCRIKQIINMWQEVGRAMYAPPIEGNITCKSNITGLLLVRDGGTEANTTETFRPGGGDMRNNWRSELYKYKVVEIKPLGVVPTEAKRRVVEREKRAVGIGAVFLGFLGAAGSTMGAASITLTAQARQLLSGIVQQQSNLLRAIEAQQHLLQLTVWGIKQLQTRVLAIERYLKDQQLLGIWGCSGKLICTTAVPWNSSWSNRSQEEIWNNMTWMQWDREISNYTNTIYRLLEDSQNQQEKNEKDLLALDSWKNLWSWFDITNWLWYIKIFIMIVGGLIGLRIIFAVLSIVNRVRQGYSPLSFQTLTPNPGGHDRLGRIEEEGGEQDKNRSIRLVNGFLALAWDDLRNLCLFSYHRLRDFILVIARVVELLGRNSLRGLQKGWEGLKYLGSLVQYWGLELKKSAISLFDIIAIAVAEGTDRIIELVQGICRAIRNIPRRIRQGFEAALQ

SEQ ID NO: 14 - DNA, 2532 bp, Human immunodeficiency virus type 1
atgagagtgagggggacactgaggaattatcaacaatggtggatatggggcgtcttaggcttttggatgttaatgatttgtaatgtgggaggaaacttgtgggtcacagtctattatggggtacctgtgtggaaagaagcaaaaactactctattctgtgcatcagatgctaaagcacatgagagagaggtgcataatgtctgggctacacatgcctgtgtacccacagaccccaacccacaagaaatggttttggaaaatgtaacagaaaattttaacatgtggaaaaatgacatggtgaatcagatgcatgaggatgtaatcagtttatgggatcaaagcctaaagccatgtgtaaagttgaccccactctgtgtccctttaaaatgtaaaaatgttacctacaatgagagtatgcaggaaataaaaaattgctctttcaatgcaaccacagatttaagagataggaagcagacagtgcaggcactcttttataaacttgatatagtatcacttaatgagaagaactctagtgagtattatagattaataaattgtaatacctcagccataacacaagcctgtccaaaggtcacttttgatccaatccctatacattattgtactccggctggttatgcgattctaaagtgtaatgagcagacattcaatgggacaggaccatgccataatgttagcacagtacaatgtacacatggaattaagccagtagtatcaactcaactactgttaaatggtagcctagcagaaagagagataataattagatctgaaaatttgacaaacaatgtcaaaacaataatagtacatcttaatcaatctgtagaaattgtgtgtacaagacccaacaataatacaagaaaaagtataaggataggaccaggacaaacattctatgcaacaggagacataatgggagacataagacaagcacattgtaacattagtgcaggaaaatggaatgaaactttacaaagggtaggtaacaaattagcagaacacttccctaataaaacaataaaatttgcaccatcttcaggaggggacctagaaattacaacacatagctttaattgtagaggagaatttttctattgtaatacatcaggcctgtttaatggtacatacaattggacagaaagtaattcaagctcaatcatcacaatcccatgcagaataaagcaaattataaacatgtggcaggaggtaggacgagcaatgtatgcccctcccattgaaggaaacataacatgcaaatcaaatatcacaggactactattggtacgtgatggaggaacagaggcaaatacgacagagacattcagacctggaggaggagatatgaggaacaattggagaagtgaattatataaatataaagtggtagaaattaagccattgggagtagtacccacagaagcaaaaaggagagtggtggagagagaaaaaagagcagtgggaataggagctgtgttccttgggttcttgggagcagcaggaagcactatgggcgcggcgtcaataacgctgacggcacaggccagacaattgttgtctggtatagtgcaacagcaaagcaatttgctgagggctatagaagcgcaacagcatctgttgcagctcacggtctggggcattaagcagctccagacaagagtcctggctatagaaagatacctaaaggatcaacagctcctagggatttggggctgctctggaaaactcatctgcactactgctgtaccttggaactccagttggagtaacagatctcaagaagagatttggaataacatgacctggatgcagtgggatagagaaattagtaattacacaaacacaatatacaggttgcttgaagactcgcaaaaccagcaggaaaaaaatgaaaaggatttattagcattggacagttggaaaaatctatggagttggtttgacataacaaattggctgtggtatataaaaatattcataatgatagtaggaggcttgataggtttaagaataatttttgctgtgctctctatagtgaatagagttaggcagggatactcacctttgtcgtttcagacccttaccccgaacccagggggacacgacaggctcggaagaatcgaagaagaaggtggagagcaagacaaaaacagatccattcgattagtgaacggattcttagcacttgcctgggacgatctgcggaacctgtgcctcttcagctaccaccgattgagagacttcatattagtgatagcgagagtggtggaacttctgggacgcaacagtctcaggggactacagaagggatgggaaggccttaaatatctgggaagtcttgtgcagtattggggtctggagctaaaaaagagtgctattagtctgtttgatatcatagcaatagcagtagctgaaggaacagatagaattatagaattagtacaaggaatttgtagagctatccgcaacatacctagaagaataagacagggctttgaagcagctttgcaataa

SEQ ID NO: 15 - protein, 169 aa, Human immunodeficiency virus type 1
MEPVDPNLEPWNHPGSQPKTACNNCACKHCSAHCLVCFQKKGLGISYGRKKRRQRRRAPQSSEDHQNLISKQMAGRSGDSDEALLRAVRIIKILYQSNPYPEPRGTRQARKNRRRRWRARQKQIHSLSERILSTCLGRSAEPVPLQDLESGGTSGTQQSQGTTEGVGSP

SEQ ID NO: 16 - protein, 769 aa, Human immunodeficiency virus type 1
MGGKWSKCSIVGWPAVRERMRRTEPAAEGVGAASQDLDKYGALTSSNTDTTNADCAWLRAQAAAAAVGFPVRPQVPLRPMTFKGAFDLSFFLKEKGGLEGLIYSKKRQEILDLWVYHTQGFFPDWQNYTPGPGVRFPLTFGWCFKLVPVDPREVEEANEGENNCLLHPVCQHGMEDEHREVLQWKFDSHLAHRHMARELHPEYYKDCMGPISPIETVPVKLKPGMDGPKVKQWPLTEEKIKALTAICDEMEKEGKITKIGPENPYNTPIFAIKKKDSTKWRKLVDFRELNKRTQDFWEVQLGIPHPAGLKKKKSVTVLDVGDAYFSVPLYEDFRKYTAFTIPSVNNETPGIRYQYNVLPQGWKGSPAIFQCSMTRILEPFRAQNPEIVIYQYMNNLYVGSDLEIGQHRAKIEELREHLLRWGFTTPDKKHQKEPPFLWMGYELHPDKWTVQPIQLPEKDSWTVNDIQKLVGKLNWASQIYPGIKVRQLCKLLRGAKTLTDIVPLTEEAELELAENREILKEPVHGVYYDPSKDLIAEIQKQGQDQWTYQIYQEPFKNLKTGKYAKMRTAHTNDVKQLTEAVQKIAMESIVIWGKTPKFRLPIQKETWETWWTDYWQATWIPEWEFVNTPPLVKLWYQLEKDPIAGVETFYVDGAANRDTKIGKAGYVTDRGRQKIVSLTETTNQKTELQAICLALQDSGSEVNIVTDSQYALGIIQAQPDKSESELVNQIIEQLIKKERVYLSWVPAHKGIGGNEQVDKLVSNGIRKVL

SEQ ID NO: 17 - protein, 72 aa, Human immunodeficiency virus type 1
MEPVDPNLEPWNHPGSQPKTACNNCACKHCSAHCLVCFQKKGLGISYGRKKRRQRRRAPQSSEDHQNLISKQ

SEQ ID NO: 18 - protein, 107 aa, Human immunodeficiency virus type 1
MAGRSGDSDEALLRAVRIIKILYQSDPYPEPRGTRQARKNRRRRWRARQKQIHSLSERILSTCLGRSAEPVPLQLPPLERLHISGSESGGTSGTQQSQGTTEGVGSP

SEQ ID NO: 19 - protein, 97 aa, Human immunodeficiency virus type 1
MAGRSGDSDEALLRAVRIIKILYQSDPYPEPRGTRQARKNRRRRWRARQKQIHSLSERILSTCLGRSAEPVPLQDLESGGTSGTQQSQGTTEGVGSP

SEQ ID NO: 20 - protein, 330 aa, Human immunodeficiency virus type 1
VQLGIPHPAGLKKKKSVTVLDVGDAYFSVPLYEDFRKYTAFTIPSVNNETPGIRYQYNVLPQGWKGSPAIFQCSMTRILEPFRAQNPEIVIYQYMDDLYVGSDLEIGQHRAKIEELREHLLRWGFTTPDKKHQKEPPFLWMGYELHPDKWTVQPIQLPEKDSWTVNDIQKLVGKLNWASQIYPGIKVRQLCKLLRGAKTLTDIVPLTEEAELELAENREILKEPVHGVYYDPSKDLIAEIQKQGQDQWTYQIYQEPFKNLKTGKYAKMRTAHTNDVKQLTEAVQKIAMESIVIWGKTPKFRLPIQKETWETWWTDYWQATWIPEWEFVNT

SEQ ID NO: 21 - protein, 330 aa, Human immunodeficiency virus type 1
VQLGIPHPAGLKKKKSVTVLDVGDAYFSVPLYEDFRKYTAFTIPSVNNETPGIRYQYNVLPQGWKGSPAIFQCSMTRILEPFRAQNPEIVIYQYMNNLYVGSDLEIGQHRAKIEELREHLLRWGFTTPDKKHQKEPPFLWMGYELHPDKWTVQPIQLPEKDSWTVNDIQKLVGKLNWASQIYPGIKVRQLCKLLRGAKTLTDIVPLTEEAELELAENREILKEPVHGVYYDPSKDLIAEIQKQGQDQWTYQIYQEPFKNLKTGKYAKMRTAHTNDVKQLTEAVQKIAMESIVIWGKTPKFRLPIQKETWETWWTDYWQATWIPEWEFVNT

SEQ ID NO: 22 - protein, 207 aa, Human immunodeficiency virus type 1
MGGKWSKCSIVGWPAVRERMRRTEPAAEGVGAASQDLDKYGALTSSNTDTTNADCAWLRAQAAAAAVGFPVRPQVPLRPMTFKGAFDLSFFLKEKGGLEGLIYSKKRQEILDLWVYHTQGFFPDWQNYTPGPGVRFPLTFGWCFKLVPVDPREVEEANEGENNCLLHPVCQHGMEDEHREVLQWKFDSHLAHRHMARELHPEYYKDC

SEQ ID NO: 23 - protein, 12 aa, Artificial Sequence
  Description of Artificial Sequence Synthetic peptide
LLPLERLHISGS

SEQ ID NO: 24 - protein, 9 aa, Artificial Sequence
  Description of Artificial Sequence Synthetic peptide
TYNETYNEI

SEQ ID NO: 25 - protein, 9 aa, Artificial Sequence
  Description of Artificial Sequence Synthetic peptide
AMQMLKDTI

SEQ ID NO: 26 - protein, 9 aa, Artificial Sequence
  Description of Artificial Sequence Synthetic peptide
YYDPSKDLI

SEQ ID NO: 27 - protein, 16 aa, Artificial Sequence
  Description of Artificial Sequence Synthetic peptide
SNGTYNETYNEIKNCS

SEQ ID NO: 28 - protein, 15 aa, Artificial Sequence
  Description of Artificial Sequence Synthetic peptide
HQAAMQMLKDTINEE

SEQ ID NO: 29 - protein, 14 aa, Artificial Sequence
  Description of Artificial Sequence; Synthetic peptide
VHGAYVPSKDLIAE

(19) United States
(12) Patent Application Publication
(10) Pub. No.: US 2008/0274992 A1
(43) Pub. Date: Nov. 6, 2008

(54) RECOMBINANT MODIFIED ANKARA VIRAL HIV-1 VACCINES

(76) Inventors: Jean-Louis Excler, Trelex (CH); Patricia E. Fast, New York, NY (US)

Correspondence Address: FROMMER LAWRENCE & HAUG, 745 FIFTH AVENUE- 10TH FL., NEW YORK, NY, 10151, US

(21) Appl. No.: 12/055,619

(22) Filed: Mar. 26, 2008

Related U.S. Application Data
(60) Provisional application No. 60/908,082, filed on Mar. 26, 2007.

(51) Int. Cl. A61K 31/711; A61P 31/18

(52) U.S. Cl. 514/44

(57) ABSTRACT

The field of the present invention relates to novel recombinant MVA vectors encoding one or more HIV-1 immunogens as an HIV-1 vaccine candidate and methods of using same.

### INCORPORATION BY REFERENCE

This application claims priority to U.S. provisional patent application Ser. No. 60/908,082 filed Mar. 26, 2007.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The field of the present invention relates to novel recombinant modified Ankara viral vectors (MVA) encoding HIV-1 antigens for use as HIV-1 vaccines.

### BACKGROUND OF THE INVENTION

AIDS, or Acquired Immunodeficiency Syndrome, is caused by human immunodeficiency virus (HIV) and is characterized by several clinical features including wasting syndromes, central nervous system degeneration and profound immunosuppression that results in opportunistic infections and malignancies. HIV is a member of the lentivirus family of animal retroviruses, which include the visna virus of sheep and the bovine, feline, and simian immunodeficiency viruses (SIV). Two closely related types of HIV, designated HIV-1 and HIV-2, have been identified thus far, of which HIV-1 is by far the most common cause of AIDS. However, HIV-2, which differs in genomic structure and antigenicity, causes a similar clinical syndrome.

An infectious HIV particle consists of two identical strands of RNA, each approximately 9.2 kb long, packaged within a core of viral proteins. This core structure is surrounded by a phospholipid bilayer envelope derived from the host cell membrane that also includes virally-encoded membrane proteins (Abbas et al., Cellular and Molecular Immunology, 4th edition, W.B. Saunders Company, 2000, p. 454). The HIV genome has the characteristic 5′-LTR-Gag-Pol-Env-LTR-3′ organization of the retrovirus family. Long terminal repeats (LTRs) at each end of the viral genome serve as binding sites for transcriptional regulatory proteins from the host and regulate viral integration into the host genome, viral gene expression, and viral replication.

The HIV genome encodes several structural proteins. The Gag gene encodes core structural proteins of the nucleocapsid core and matrix. The Pol gene encodes reverse transcriptase (RT), integrase (Int), and viral protease enzymes required for viral replication. The tat gene encodes a protein that is required for elongation of viral transcripts. The rev gene encodes a protein that promotes the nuclear export of incompletely spliced or unspliced viral RNAs. The Vif gene product enhances the infectivity of viral particles. The vpr gene product promotes the nuclear import of viral DNA and regulates G2 cell cycle arrest. The vpu and nef genes encode proteins that down regulate host cell CD4 expression and enhance release of virus from infected cells. The Env gene encodes the viral envelope glycoprotein that is translated as a 160-kilodalton (kDa) precursor (gp160) and cleaved by a cellular protease to yield the external 120-kDa envelope glycoprotein (gp120) and the transmembrane 41-kDa envelope glycoprotein (gp41), which are required for the infection of cells (Abbas, pp. 454-456). Gp140 is a modified form of the env glycoprotein which contains the external 120-kDa envelope glycoprotein portion and a part of the gp41 portion of env and has characteristics of both gp120 and gp41. The Nef gene is conserved among primate lentiviruses and is one of the first viral genes that is transcribed following infection. In vitro, several functions have been described, including down regulation of CD4 and MHC class I surface expression, altered T-cell signaling and activation, and enhanced viral infectivity. The HIV-1 transactivator of transcription (Tat) protein is a pleiotropic factor that induces a broad range of biological effects in numerous cell types. At the HIV promoter, Tat is a powerful transactivator of gene transcription, which acts by both inducing chromatin remodeling and by recruiting elongation-competent transcriptional complexes onto the vital LTR. Besides these transcriptional activities, Tat is released outside of the cells and interacts with different cell membrane-associated receptors. Finally, extracellular Tat can be externalized by cells through an active endocytosis process.

HIV infection initiates with gp120 on the viral particle binding to the CD4 and chemokine receptor molecules (e.g., CXCR4, CCR5) on the cell membrane of target cells such as CD4+ T-cells, macrophages and dendritic cells. The bound virus fuses with the target cell and reverse transcribes the RNA genome. The resulting viral DNA integrates into the cellular genome, where it directs the production of new viral RNA, and thereby viral proteins and new virions. These virions bud from the infected cell membrane and establish productive infections in other cells. This process also kills the originally infected cell. HIV can also kill cells indirectly because the CD4 receptor on uninfected T-cells has a strong affinity for gp120 expressed on the surface of infected cells. In this case, the uninfected cells bind, via the CD4 receptor-gp120 interaction, to infected cells and fuse to form a syncytium, which cannot survive. Destruction of CD4+ T-lymphocytes, which are critical to immune defense, is a major cause of the progressive immune dysfunction that is the hallmark of AIDS disease progression. The loss of CD4+ T cells seriously impairs the body's ability to fight most invaders, but it has a particularly severe impact on the defenses against viruses, fungi, parasites and certain bacteria, including mycobacteria.

Research on the Env glycoproteins have shown that the virus has many effective protective mechanisms with few vulnerabilities (Wyatt & Sodroski, Science. 1998 Jun. 19; 280(5371):1884-8). For fusion with its target cells, HIV-1 uses a trimeric Env complex containing gp120 and gp41 subunits (Burton et al., Nat. Immunol. 2004 March; 5(3):233-6). The fusion potential of the Env complex is triggered by engagement of the CD4 receptor and a receptor, usually CCR5 or CXCR4. Neutralizing antibodies seem to work either by binding to the mature trimer on the virion surface and preventing initial receptor engagement events or by binding after virion attachment and inhibiting the fusion process (Parren & Burton, Adv Immunol. 2001; 77:195-262). In the latter case, neutralizing antibodies may bind to epitopes whose exposure is enhanced or triggered by receptor binding. However, given the potential antiviral effects of neutralizing antibodies, it is not unexpected that HIV-1 has evolved multiple mechanisms to protect it from antibody binding (Johnson & Desrosiers, Annu Rev Med. 2002; 53:499-518).

Accordingly, there remains a need for efficacious immunization again HIV-1.

Citation or identification of any document in this application is not an admission that such document is available as prior art to the present invention.

### SUMMARY OF THE INVENTION

The present invention is directed to a recombinant MVA vaccine for the induction of an immune response to the target HIV-1 proteins inserted into a MVA viral vector. All six selected HIV proteins (env, gag, nef, reverse transcriptase (RT), tat and rev) are expressed by the recombinant MVA virus.

The recombinant MVA vaccine of the present invention elicits a high immunogenicity response rate in Phase I studies and therefore may be an efficacious vaccine against HIV infection.

The present invention relates to method for obtaining an immunogenic response which may comprise administering to a mammal: an immunological composition against one or more immunogens comprising a MVA containing and expressing a nucleotide sequence encoding one or more immunogens.

The present invention also relates to method for obtaining an immunogenic response which may comprise administering to a mammal: (a) an immunological composition against a first immunogen comprising a MVA containing and expressing a nucleotide sequence encoding one or more immunogens; and (b) an immunological composition against one or more immunogens comprising a MVA containing and expressing a nucleotide sequence encoding the second immunogen of a pathogen of the mammal, wherein (a) and (b) are administered sequentially. The one or more immunogens administered first and second may be the same one or more immunogens or different one or more immunogens.

In an advantageous embodiment, the one or more immunogens is selected from the group consisting of HIV proteins encoded by the env, gag, nef, reverse transcriptase (RT), tat and rev genes, or a fragment thereof.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including” and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 illustrates the plasmid construct/genomic structure of TBC-M4;

FIGS. 2A-2C depict the sequence of TB19a.1, the 49/50 insertion region;

FIGS. 2D-2G depict the sequence of TB19a.2, the del III insertion region;

FIG. 3A depicts sequences of nef,

FIGS. 3B-3C depict sequences of rev;

FIG. 3D depicts sequences of gag;

FIG. 3E depicts sequences of tat;

FIGS. 3F-3G depict sequences of pol;

FIGS. 3H-31 depict sequences of env;

FIG. 4A depicts the predicted amino acid sequence of env;

FIG. 4B depicts the predicted amino acid sequence of gag;

FIG. 4C depicts the predicted amino acid sequence of tat.rev;

FIG. 4D depicts the predicted amino acid sequence of nef.RT;

FIG. 5A depicts the sequence alignment of natural/wild type vs. modified amino acid sequence of tat;

FIG. 5B depicts the sequence alignment of natural/wild type vs. modified amino acid sequence of rev;

FIG. 5C depicts the sequence alignment of natural/wild type vs. modified amino acid sequence of RT;

FIG. 5D depicts the sequence alignment of natural/wild type vs. modified amino acid sequence of nef;

FIG. 6 depicts an annotated plasmid map of a transfer vector and

FIG. 7 depicts a flow chart outlining the isolation of the TBC-M420 recombinant and the preparation of the seed stock.

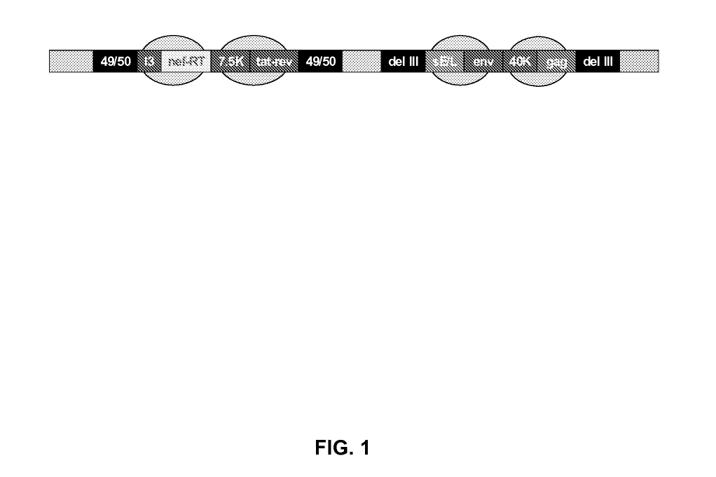

### DETAILED DESCRIPTION

The present invention relates to method for obtaining an immunogenic response which may comprise administering to a mammal: an immunological composition against one or more immunogens comprising a MVA containing and expressing a nucleotide sequence encoding one or more immunogens.

The present invention also relates to method for obtaining an immunogenic response which may comprise administering to a mammal: (a) an immunological composition against a first immunogen comprising a MVA containing and expressing a nucleotide sequence encoding one or more immunogens; and (b) an immunological composition against one or more immunogens comprising a MVA containing and expressing a nucleotide sequence encoding the second immunogen of a pathogen of the mammal, wherein (a) and (b) are administered sequentially. The one or more immunogens administered first and second may be the same one or more immunogens or different one or more immunogens.

The terms “protein”, “peptide”, “polypeptide”, and “amino acid sequence” are used interchangeably herein to refer to polymers of amino acid residues of any length. The polymer may be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component.

As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

It should be understood that the proteins and antigens of the invention may differ from the exact sequences illustrated and described herein. Thus, the invention contemplates deletions, additions and substitutions to the sequences shown, so long as the sequences function in accordance with the methods of the invention. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cystine, serine threonine, tyrosine. Phenylalanine, tryptophan, and tyrosine are sometimes classified as aromatic amino acids. It is reasonably predictable that an isolated replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the sequences illustrated and described but possessing minor amino acid substitutions that do not substantially affect the immunogenicity of the protein are, therefore, within the scope of the invention.

In an advantageous embodiment, the immunogens of the present invention are HIV-1 proteins, advantageously HIV-1 proteins encoded by the env, gag, nef, reverse transcriptase (RT), tat and rev genes, or any immunogenic fragment thereof. In an advantageous embodiment, env and RT sequences are derived from GenBank Accession No. AF067158 (see, e.g., Lole et al., J. Virol. 1999 January; 73(1):152-60, the disclosure of which is incorporated by reference), gag and tat sequences are derived from GenBank Accession No. AF067157 (see, e.g., Lole et al., J. Virol. 1999 January; 73(1):152-60, the disclosure of which is incorporated by reference), and rev and nef sequences are derived from GenBank Accession No. AF067154 (see, e.g., Lole et al., J. Virol. 1999 January; 73(1):152-60, the disclosure of which is incorporated by reference).

In a particularly advantageous embodiment, the TBC-M4 HIV gene sequence insert encodes the immunogens of the present invention.

As used herein the terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid can be single-stranded, or partially or completely double-stranded (duplex). Duplex nucleic acids can be homoduplex or heteroduplex.

As used herein the term “transgene” is used to refer to “recombinant” nucleotide sequences that are derived from sequences of HIV-1 antigens known to one of skill in the art. The sequence of transgenes may be derived from either the HIV-1 Clade A consensus nucleotide sequences of the invention, or from the nucleotide sequences that encode the antigens from recently circulating HIV-1 Clade A strains that have been identified as being closely matched to these consensus sequences. The term “recombinant” means a nucleotide sequence that has been manipulated “by man” and which does not occur in nature, or is linked to another nucleotides sequence or found in a different arrangement in nature. It is understood that manipulated “by man” means manipulated by some artificial means, including by use of machines, codon optimization, restriction enzymes, etc.

The nucleotides of the invention may be altered as compared to the consensus nucleotide sequences, or as compared to the sequences from circulating HIV-1 isolates that are closely related to such consensus sequences. For example, in one embodiment the nucleotide sequences may be mutated such that the activity of the encoded proteins in vivo is abrogated. In another embodiment the nucleotide sequences may be codon optimized, for example the codons may be optimized for human use. In preferred embodiments the nucleotide sequences of the invention are both mutated to abrogate the normal in vivo function of the encoded proteins, and codon optimized for human use. For example, each of the Gag, Pol, Env, Nef, RT, Tat and Rev sequences of the invention may be altered in these ways.

In a particularly advantageous embodiment, the target HIV-1 subtype C genes were modified as follows:

Full-length env is modified to introduce silent mutations to internal T₅NT motifs that encode early transcription termination signals for vaccinia virus as elimination of the T₅NT sequences is known to minimize premature transcription termination and optimize foreign gene expression in vaccinia virus.

Full length gag gene encoding the p55 poly-protein is isolated without any modifications.

The rev gene is modified in several ways. Twelve codons, encoding amino acids 75-86, were deleted and replaced with two codons, encoding aspartic acid and leucine, to render the rev protein non-functional. In addition, the nucleotide sequence of the rev gene is altered at codon position 3 (“wobbled”) to minimize homology between the tat and rev genes and to optimize expression of rev protein in human cells, “humanize” expression, without otherwise changing the amino acid sequence.

The first exon of the tat gene is modified by in vitro mutagenesis to change two codons, at amino acids 26 and 32, from tyrosine to alanine, to render the protein nonfunctional while preserving the 3-dimensional structure. In addition, the second exon of the tat gene is deleted.

The modified tat and rev sequences are cloned as a fusion gene, with appropriate initiation and termination codons.

The nef gene is modified by changing codons at amino acids 62-65 from glutamic acid to alanine to reduce MHC class I downregulation and CD3 signaling.

The reverse transcriptase (RT) portion of the pol gene is modified by changing codons at amino acids 336 and 337 from aspartic acid to aspargine to eliminate reverse transcriptase activity. Protease and integrase sequences are not included in the construct.

The modified nef and RT coding sequences are fused in frame to form a nef-RT fusion gene.

The types of mutations that can be made to abrogate the in vivo function of the antigens. Mutation of Gly2 to Ala in Gag to remove a myristylation site and prevent formation of virus-like-particles (VLPs); Mutation of Gag to avoid slippage at the natural frame shift sequence to leave the conserved amino acid sequence (NFLG) intact and allow only the full-length GagPol protein product to be translated; Mutation of RT Asp 185 to Ala and mutation of Asp 186 to Ala to inactivate active enzyme residues. Mutation of Int Asp 64 to Ala, and mutation of Asp 116 to Ala and mutation of Glu 152 to Ala to inactivate active enzyme residues.

As regards codon optimization, the nucleic acid molecules of the invention have a nucleotide sequence that encodes the antigens of the invention and can be designed to employ codons that are used in the genes of the subject in which the antigen is to be produced. Many viruses, including HIV and other lentiviruses, use a large number of rare codons and, by altering these codons to correspond to codons commonly used in the desired subject, enhanced expression of the antigens can be achieved. In a preferred embodiment, the codons used are “humanized” codons, i.e., the codons are those that appear frequently in highly expressed human genes (Andre et al., J. Virol. 72:1497-1503, 1998) instead of those codons that are frequently used by HIV. Such codon usage provides for efficient expression of the transgenic HIV proteins in human cells. Any suitable method of codon optimization may be used. However, any other suitable methods of codon optimization may be used. Such methods, and the selection of such methods, are well known to those of skill in the art. In addition, there are several companies that will optimize codons of sequences, such as Geneart (geneart.com). Thus, the nucleotide sequences of the invention can readily be codon optimized.

The invention further encompasses nucleotide sequences encoding functionally and/or antigenically equivalent variants and derivatives of the antigens of the invention and functionally equivalent fragments thereof. These functionally equivalent variants, derivatives, and fragments display the ability to retain antigenic activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan. In one embodiment, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the antigen, epitope, immunogen, peptide or polypeptide of interest.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms can be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

The various recombinant nucleotide sequences and immunogens of the invention are made using standard recombinant DNA and cloning techniques. Such techniques are well known to those of skill in the art. See for example, “Molecular Cloning: A Laboratory Manual”, second edition (Sambrook et al. 1989).

The nucleotide sequences of the present invention may be inserted into “vectors.” The term “vector” is widely used and understood by those of skill in the art, and as used herein the term “vector” is used consistent with its meaning to those of skill in the art. For example, the term “vector” is commonly used by those skilled in the art to refer to a vehicle that allows or facilitates the transfer of nucleic acid molecules from one environment to another or that allows or facilitates the manipulation of a nucleic acid molecule.

Any vector that allows expression of the immunogens of the present invention may be used in accordance with the present invention. In certain embodiments, the immunogens of the present invention may be used in vitro (such as using cell-free expression systems) and/or in cultured cells grown in vitro in order to produce the encoded HIV-1 antigens which may then be used for various applications such as in the production of proteinaceous vaccines. For such applications, any vector that allows expression of the immunogens in vitro and/or in cultured cells may be used.

For applications where it is desired that the immunogens be expressed in vivo, for example when the immunogens of the invention are used in DNA or DNA-containing vaccines, any vector that allows for the expression of the immunogens of the present invention and is safe for use in vivo may be used. In preferred embodiments the vectors used are safe for use in humans, mammals and/or laboratory animals.

In order for the immunogens of the present invention to be expressed, the protein coding sequence should be “operably linked” to regulatory or nucleic acid control sequences that direct transcription and translation of the protein. As used herein, a coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” can be any nucleic acid element, such as, but not limited to promoters, enhancers, IRES, introns, and other elements described herein that direct the expression of a nucleic acid sequence or coding sequence that is operably linked thereto. The term “promoter” will be used herein to refer to a group of transcriptional control modules that are clustered around the initiation site for RNA polymerase II and that when operationally linked to the protein coding sequences of the invention lead to the expression of the encoded protein. The expression of the immunogens of the present invention can be under the control of a constitutive promoter or of an inducible promoter, which initiates transcription only when exposed to some particular external stimulus, such as, without limitation, antibiotics such as tetracycline, hormones such as ecdysone, or heavy metals. The promoter can also be specific to a particular cell-type, tissue or organ. Many suitable promoters and enhancers are known in the art, and any such suitabel promoter or enhancer may be used for expression of the immunogens of the invention. For example, suitable promoters and/or enhancers can be selected from the Eukaryotic Promoter Database (EPDB).

The vectors used in accordance with the present invention should typically be chosen such that they contain a suitable gene regulatory region, such as a promoter or enhancer, such that the immunogens of the invention can be expressed.

For example, when the aim is to express the immunogens of the invention in vitro, or in cultured cells, or in any prokaryotic or eukaryotic system for the purpose of producing the protein(s) encoded by that immunogen, then any suitable vector can be used depending on the application. For example, plasmids, viral vectors, bacterial vectors, protozoal vectors, insect vectors, baculovirus expression vectors, yeast vectors, mammalian cell vectors, and the like, can be used. Suitable vectors can be selected by the skilled artisan taking into consideration the characteristics of the vector and the requirements for expressing the immunogens under the identified circumstances.

When the aim is to express the immunogens of the invention in vivo in a subject, for example in order to generate an immune response against an HIV-1 antigen and/or protective immunity against HIV-1, expression vectors that are suitable for expression on that subject, and that are safe for use in vivo, should be chosen. For example, in some embodiments it may be desired to express the immunogens of the invention in a laboratory animal, such as for pre-clinical testing of the HIV-1 immunogenic compositions and vaccines of the invention. In other embodiments, it will be desirable to express the immunogens of the invention in human subjects, such as in clinical trials and for actual clinical use of the immunogenic compositions and vaccine of the invention. Any vectors that are suitable for such uses can be employed, and it is well within the capabilities of the skilled artisan to select a suitable vector. In some embodiments it may be preferred that the vectors used for these in vivo applications be attenuated to prevent vector from amplifying in the subject. For example, if plasmid vectors are used, preferably they will lack an origin of replication that functions in the subject so as to enhance safety for in vivo use in the subject. If viral vectors are used, preferably they are attenuated or replication-defective in the subject, again, so as to enhance safety for in vivo use in the subject.

In preferred embodiments of the present invention viral vectors are used. Viral expression vectors are well known to those skilled in the art and include, for example, viruses such as adenoviruses, adeno-associated viruses (AAV), alphaviruses, retroviruses and poxviruses, including avipox viruses, attenuated poxviruses, vaccinia viruses, and particularly, the modified vaccinia Ankara virus (MVA; ATCC Accession No. VR-1566). Such viruses, when used as expression vectors are innately non-pathogenic in the selected subjects such as humans or have been modified to render them non-pathogenic in the selected subjects. For example, replication-defective adenoviruses and alphaviruses are well known and can be used as gene delivery vectors.

In particularly preferred embodiments MVA vectors are used. MVA is a live attenuated strain derived from wild type vaccinia virus through chick embryo fibroblast (CEF) cells. During the attenuation process, the MVA virus underwent multiple well-characterized genomic deletions that have been associated with reduced pathogenicity. The genomic deletions have been extensively characterized and appear to affect late stage virion assembly and expression of cytokine receptors. As a consequence, the modified virus infects most mammalian (including human) cells and to express viral (and recombinant) genes in a normal way, but does not replicate efficiently in most primary cell types or immortalized cell lines. The MVA vectors of any of U.S. Pat. Nos. 7,189,536; 7,118,754; 7,097,842; 7,094,412; 7,067,251; 7,056,723; 7,049,145; 7,034,141; 6,960,345; 6,924,137; 6,913,752; 6,893,869; 6,884,786; 6,869,793; 6,663,871; 6,649,409; 6,582,693; 6,440,422; 5,676,950 and 5,185,146 may be utilized and/or modified for the present invention.

In an advantageous embodiment, the MVA of the present invention is derived from an attenuated MVA.

The nucleotide sequences and vectors of the invention can be delivered to cells, for example if the aim is to express the HIV-1 antigens in cells to produce and isolate the expressed proteins, such as from cells grown in culture. For expressing the antigens in cells any suitable transfection, transformation, or gene delivery methods can be used. Such methods are well known by those skilled in the art, and one of skill in the art would readily be able to select a suitable method depending on the nature of the nucleotide sequences, vectors, and cell types used. For example, transfection, transformation, microinjection, infection, electroporation, lipofection, or liposome-mediated delivery could be used. Expression of the antigens can be carried out in any suitable type of host cells, such as bacterial cells, yeast, insect cells, and mammalian cells. The HIV-1 antigens of the invention can also be expressed using in vitro transcription/translation systems. All of such methods are well known by those skilled in the art, and one of skill in the art would readily be able to select a suitable method depending on the nature of the nucleotide sequences, vectors, and cell types used.

Following expression, the antigens of the invention can be isolated and/or purified or concentrated using any suitable technique known in the art. For example, anion or cation exchange chromatography, phosphocellulose chromatography, hydrophobic interaction chromatography, affinity chromatography, immuno-affinity chromatography, hydroxyapatite chromatography, lectin chromatography, molecular sieve chromatography, isoelectric focusing, gel electrophoresis, or any other suitable method or combination of methods can be used.

In preferred embodiments, the nucleotide sequences and/or antigens of the invention are administered in vivo, for example where the aim is to produce an immunogenic response in a subject. A “subject” in the context of the present invention may be any animal. For example, in some embodiments it may be desired to express the immunogens of the invention in a laboratory animal, such as for pre-clinical testing of the HIV-1 immunogenic compositions and vaccines of the invention. In other embodiments, it will be desirable to express the immunogens of the invention in human subjects, such as in clinical trials and for actual clinical use of the immunogenic compositions and vaccine of the invention. In preferred embodiments the subject is a human, for example a human that is infected with, or is at risk of infection with, HIV-1.

For such in vivo applications the nucleotide sequences and/or antigens of the invention are preferably administered as a component of an immunogenic composition comprising the nucleotide sequences and/or antigens of the invention in admixture with a pharmaceutically acceptable carrier. The immunogenic compositions of the invention are useful to stimulate an immune response against HIV-1 and may be used as one or more components of a prophylactic or therapeutic vaccine against HIV-1 for the prevention, amelioration or treatment of AIDS. The nucleic acids and vectors of the invention are particularly useful for providing genetic vaccines, i.e. vaccines for delivering the nucleic acids encoding the antigens of the invention to a subject, such as a human, such that the antigens are then expressed in the subject to elicit an immune response.

The compositions of the invention may be injectable suspensions, solutions, sprays, lyophilized powders, syrups, elixirs and the like. Any suitable form of composition may be used. To prepare such a composition, a nucleic acid or vector of the invention, having the desired degree of purity, is mixed with one or more pharmaceutically acceptable carriers and/or excipients. The carriers and excipients must be “acceptable” in the sense of being compatible with the other ingredients of the composition. Acceptable carriers, excipients, or stabilizers are nontoxic to recipients at the dosages and concentrations employed, and include, but are not limited to, water, saline, phosphate buffered saline, dextrose, glycerol, ethanol, or combinations thereof, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

An immunogenic or immunological composition can also be formulated in the form of an oil-in-water emulsion. The oil-in-water emulsion can be based, for example, on light liquid paraffin oil (European Pharmacopea type); isoprenoid oil such as squalane, squalene, EICOSANE™ or tetratetracontane; oil resulting from the oligomerization of alkene(s), e.g., isobutene or decene; esters of acids or of alcohols containing a linear alkyl group, such as plant oils, ethyl oleate, propylene glycol di(caprylate/caprate), glyceryl tri(caprylate/caprate) or propylene glycol dioleate; esters of branched fatty acids or alcohols, e.g., isostearic acid esters. The oil advantageously is used in combination with emulsifiers to form the emulsion. The emulsifiers can be nonionic surfactants, such as esters of sorbitan, mannide (e.g., anhydromannitol oleate), glycerol, polyglycerol, propylene glycol, and oleic, isostearic, ricinoleic, or hydroxystearic acid, which are optionally ethoxylated, and polyoxypropylene-polyoxyethylene copolymer blocks, such as the Pluronic (products, e.g., L121. The adjuvant can be a mixture of emulsifier(s), micelle-forming agent, and oil such as that which is commercially available under the name Provax® (IDEC Pharmaceuticals, San Diego, Calif.).

The immunogenic compositions of the invention can contain additional substances, such as wetting or emulsifying agents, buffering agents, or adjuvants to enhance the effectiveness of the vaccines (Remington's Pharmaceutical Sciences, 18th edition, Mack Publishing Company, (ed.) 1980).

Adjuvants may also be included. Adjuvants include, but are not limited to, mineral salts (e.g., AlK(SO₄)₂, AlNa(SO₄)₂, AlNH(SO₄)₂, silica, alum, Al(OH)₃, Ca₃(PO₄)₂, kaolin, or carbon), polynucleotides with or without immune stimulating complexes (ISCOMs) (e.g., CpG oligonucleotides, such as those described in Chuang, T. H. et al, (2002) J. Leuk. Biol. 71(3): 538-44; Ahmad-Nejad, P. et al (2002) Eur. J. Immunol. 32(7): 1958-68; poly IC or poly AU acids, polyarginine with or without CpG (also known in the art as IC₃₁; see Schellack, C. et al (2003) Proceedings of the 34^{th} Annual Meeting of the German Society of Immunology; Lingnau, K. et al (2002) Vaccine 20(29-30): 3498-508), JuvaVax™ (U.S. Pat. No. 6,693,086), certain natural substances (e.g., wax D from Mycobacterium tuberculosis, substances found in Cornyebacterium parvum, Bordetella pertussis, or members of the genus Brucella), flagellin (Toll-like receptor 5 ligand; see McSorley, S. J. et al (2002) J. Immunol. 169(7): 3914-9), saponins such as QS21, QS17, and QS7 (U.S. Pat. Nos. 5,057,540; 5,650,398; 6,524,584; 6,645,495), monophosphoryl lipid A, in particular, 3-de-O-acylated monophosphoryl lipid A (3D-MPL), imiquimod (also known in the art as IQM and commercially available as Aldara®; U.S. Pat. Nos. 4,689,338; 5,238,944; Zuber, A. K. et al (2004) 22(13-14): 1791-8), and the CCR5 inhibitor CMPD167 (see Veazey, R. S. et al (2003) J. Exp. Med. 198: 1551-1562).

Aluminum hydroxide or phosphate (alum) are commonly used at 0.05 to 0.1% solution in phosphate buffered saline. Other adjuvants that can be used, especially with DNA vaccines, are cholera toxin, especially CTA1-DD/ISCOMs (see Mowat, A. M. et al (2001) J. Immunol. 167(6): 3398-405), polyphosphazenes (Allcock, H. R. (1998) App. Organometallic Chem. 12(10-11): 659-666; Payne, L. G. et al (1995) Pharm. Biotechnol. 6: 473-93), cytokines such as, but not limited to, IL-2, IL-4, GM-CSF, IL-12, IGF-1, IFN-α, IFN-β, and IFN-γ (Boyer et al., (2002) J. Liposome Res. 121:137-142; WO01/095919), immunoregulatory proteins such as CD40L (ADX40; see, for example, WO03/063899), and the CD1a ligand of natural killer cells (also known as CRONY or α-galactosyl ceramide; see Green, T. D. et al, (2003) J. Virol. 77(3): 2046-2055), immunostimulatory fusion proteins such as IL-2 fused to the Fc fragment of immunoglobulins (Barouch et al., Science 290:486-492, 2000) and co-stimulatory molecules B7.1 and B7.2 (Boyer), all of which can be administered either as proteins or in the form of DNA, on the same expression vectors as those encoding the antigens of the invention or on separate expression vectors.

The immunogenic compositions can be designed to introduce the antigens, nucleic acids or expression vectors to a desired site of action and release it at an appropriate and controllable rate. Methods of preparing controlled-release formulations are known in the art. For example, controlled release preparations can be produced by the use of polymers to complex or absorb the immunogen and/or immunogenic composition. A controlled-release formulations can be prepared using appropriate macromolecules (for example, polyesters, polyamino acids, polyvinyl, pyrrolidone, ethylenevinylacetate, methylcellulose, carboxymethylcellulose, or protamine sulfate) known to provide the desired controlled release characteristics or release profile. Another possible method to control the duration of action by a controlled-release preparation is to incorporate the active ingredients into particles of a polymeric material such as, for example, polyesters, polyamino acids, hydrogels, polylactic acid, polyglycolic acid, copolymers of these acids, or ethylene vinylacetate copolymers. Alternatively, instead of incorporating these active ingredients into polymeric particles, it is possible to entrap these materials into microcapsules prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nano-particles and nanocapsules) or in macroemulsions. Such techniques are disclosed in New Trends and Developments in Vaccines, Voller et al. (eds.), University Park Press, Baltimore, Md., 1978 and Remington's Pharmaceutical Sciences, 16th edition.

Suitable dosages of the antigens, nucleic acids and expression vectors of the invention (collectively, the immunogens) in the immunogenic composition of the invention can be readily determined by those of skill in the art. For example, the dosage of the immunogens can vary depending on the route of administration and the size of the subject. Suitable doses can be determined by those of skill in the art, for example by measuring the immune response of a subject, such as a laboratory animal, using conventional immunological techniques, and adjusting the dosages as appropriate. Such techniques for measuring the immune response of the subject include but are not limited to, chromium release assays, tetramer binding assays, IFN-γ ELISPOT assays, IL-2 ELISPOT assays, intracellular cytokine assays, and other immunological detection assays, e.g., as detailed in the text “Antibodies: A Laboratory Manual” by Ed Harlow and David Lane.

When provided prophylactically, the immunogenic compositions of the invention are ideally administered to a subject in advance of HIV infection, or evidence of HIV infection, or in advance of any symptom due to AIDS, especially in high-risk subjects. The prophylactic administration of the immunogenic compositions can serve to provide protective immunity of a subject against HIV-1 infection or to prevent or attenuate the progression of AIDS in a subject already infected with HIV-1. When provided therapeutically, the immunogenic compositions can serve to ameliorate and treat AIDS symptoms and are advantageously used as soon after infection as possible, preferably before appearance of any symptoms of AIDS but may also be used at (or after) the onset of the disease symptoms.

The immunogenic compositions can be administered using any suitable delivery method including, but not limited to, intramuscular, intravenous, intradermal, mucosal, and topical delivery. Such techniques are well known to those of skill in the art. More specific examples of delivery methods are intramuscular injection, intradermal injection, and subcutaneous injection. However, delivery need not be limited to injection methods. Further, delivery of DNA to animal tissue has been achieved by cationic liposomes (Watanabe et al., (1994) Mol. Reprod. Dev. 38:268-274; and WO 96/20013), direct injection of naked DNA into animal muscle tissue (Robinson et al., (1993) Vaccine 11:957-960; Hoffman et al., (1994) Vaccine 12: 1529-1533; Xiang et al., (1994) Virology 199: 132-140; Webster et al., (1994) Vaccine 12: 1495-1498; Davis et al., (1994) Vaccine 12: 1503-1509; and Davis et al., (1993) Hum. Mol. Gen. 2: 1847-1851), or intradermal injection of DNA using “gene gun” technology (Johnston et al., (1994) Meth. Cell Biol. 43:353-365). Alternatively, delivery routes can be oral, intranasal or by any other suitable route. Delivery also be accomplished via a mucosal surface such as the anal, vaginal or oral mucosa.

Immunization schedules (or regimens) are well known for animals (including humans) and can be readily determined for the particular subject and immunogenic composition. Hence, the immunogens can be administered one or more times to the subject. Preferably, there is a set time interval between separate administrations of the immunogenic composition. While this interval varies for every subject, typically it ranges from 10 days to several weeks, and is often 2, 4, 6 or 8 weeks. For humans, the interval is typically from 2 to 6 weeks and up to 6 months or more. The immunization regimes typically have from 1 to 6 administrations of the immunogenic composition, but may have as few as one or two or four. The methods of inducing an immune response can also include administration of an adjuvant with the immunogens. In some instances, annual, biannual or other long interval (5-10 years) booster immunization can supplement the initial immunization protocol.

The present methods also include a variety of prime-boost regimens, especially DNA prime-adenovirus boost or DNA prime-MVA boost regimens. In these methods, one or more priming immunizations are followed by one or more boosting immunizations. The actual immunogenic composition can be the same or different for each immunization and the type of immunogenic composition (e.g., containing protein or expression vector), the route, and formulation of the immunogens can also be varied. For example, if an expression vector is used for the priming and boosting steps, it can either be of the same or different type (e.g., DNA or bacterial or viral expression vector). One useful prime-boost regimen provides for two priming immunizations, four weeks apart, followed by two boosting immunizations at 4 and 8 weeks after the last priming immunization. It should also be readily apparent to one of skill in the art that there are several permutations and combinations that are encompassed using the DNA, bacterial and viral expression vectors of the invention to provide priming and boosting regimens.

A specific embodiment of the invention provides methods of inducing an immune response against HIV in a subject by administering an immunogenic composition of the invention, preferably comprising an adenovirus vector containing DNA encoding one or more of the HIV-1 antigens of the invention, (preferably HIV proteins encoded by the env, gag, nef, reverse transcriptase (RT), tat and rev genes, or a fragment thereof), one or more times to a subject wherein the HIV-1 antigen(s) are expressed at a level sufficient to induce a specific immune response in the subject. Such immunizations can be repeated multiple times at time intervals of at least 2, 4 or 6 weeks (or more) in accordance with a desired immunization regime.

The immunogenic compositions of the invention can be administered alone, or can be co-administered, or sequentially administered, with other HIV immunogens and/or HIV immunogenic compositions, e.g., with “other” immunological, antigenic or vaccine or therapeutic compositions thereby providing multivalent or “cocktail” or combination compositions of the invention and methods of employing them. Again, the ingredients and manner (sequential or co-administration) of administration, as well as dosages can be determined taking into consideration such factors as the age, sex, weight, species and condition of the particular subject, and the route of administration.

When used in combination, the other HIV immunogens can be administered at the same time or at different times as part of an overall immunization regime, e.g., as part of a prime-boost regimen or other immunization protocol. Other HIV immunogens, such as HIV-1 transgenes (preferably GRIN, GRN, or Env, or a combination thereof) may be utilized in the present invention. Many other HIV immunogens are known in the art, one such preferred immunogen is HIVA (described in WO 01/47955), which can be administered as a protein, on a plasmid (e.g., pTHr.HIVA) or in a viral vector (e.g., MVA.HIVA). Another such HIV immunogen is RENTA (described in PCT/US2004/037699), which can also be administered as a protein, on a plasmid (e.g., pTHr.RENTA) or in a viral vector (e.g., MVA.RENTA).

For example, one method of inducing an immune response against HIV in a human subject comprises administering at least one priming dose of an HIV immunogen and at least one boosting dose of an HIV immunogen, wherein the immunogen in each dose can be the same or different, provided that at least one of the immunogens is an HIV-1 antigen of the invention, a nucleic acid encoding an HIV-1 antigen of the invention or an expression vector, preferably an adenovirus vector, encoding an HIV-1 antigen of the invention, and wherein the immunogens are administered in an amount or expressed at a level sufficient to induce an HIV-specific immune response in the subject. Advantageously, each dose is about 1×10⁷ to about 2×10¹¹ virus particles per immunization.

The HIV-specific immune response can include an HIV-specific T-cell immune response or an HIV-specific B-cell immune response. Such immunizations can be done at intervals, preferably of at least 2-6 or more weeks.

The preferred time interval between the immunization injections for prime and the boost is between about 3-6 months, advantageously six months. Preference is for single prime and then 3-6 months later single boost.

The present invention also encompasses administration of the vaccines. In a preferred embodiment, the DNA boost may be with PMED (a DNA vaccine administered with PowderJect® powder mediated epidermal delivery) technology. Advantageously, a dose of PMED is administered about 12 weeks after the homologous or heterologous boost.

It is to be understood and expected that variations in the principles of invention as described above, and as described in the below example, may be made by one skilled in the art and it is intended that such modifications, changes, and substitutions are to be included within the scope of the present invention.

The invention will now be further described by way of the following non-limiting examples.

### EXAMPLES

### Example 1

### TBC-M4 HIV Gene Sequence Insert

The TBC-M4 vaccine candidate encodes gene sequences from subtype C virus isolates. Six distinct HIV-1 isolates from India were cloned and characterized in seroconverters infected with subtype C variants. The nucleotide sequences for the isolates are available from GenBank and the viral clones are available from the National AIDS Reference Reagent Program (National Institutes of Health, USA).

A consensus sequence for each HIV-1 gene component of the candidate vaccine, namely, env, gag, RT, net tat, and rev was derived. The natural sequences from the six isolates were then compared with the derived consensus sequence to identify which isolate(s) conformed closest to the consensus sequence for each of the six target HIV-1 genes. The following isolates were determined to contain the genes that are closest to the consensus sequence:

GenBank Accession # AF067158: env and RT

GenBank Accession # AF067157: gag and tat

GenEank Accession # AF067154: rev and nef

All three of these HIV-1 isolates are subtype C and non-syncytium-inducing (NSI) phenotype. The cloned genomes of these three isolates were then obtained from the National AIDS Reference Reagent Program for the purpose of subcloning the identified target HIV-1 gene sequences.

The env, RT, gag, tat, and nef genes were subcloned from three genomic DNA clones by polymerase chain reaction (PCR) using Pfu polymerase. The rev gene was constructed from synthetic oligonucleotides due to its short length and the extensive modifications required. Nucleotide changes for the modification of the HIV-1 genes were intentionally introduced during PCR amplification by in vitro mutagenesis to optimize theoretical gene expression in the mammalian cells and to selectively reduce natural protein function.

The predicted sequence of each gene was available from GenBank. The nucleotide sequence of each subcloned gene was determined by standard genomic sequencing and was compared with the expected sequence.

The target HIV-1 subtype C genes were modified as follows:

Full-length env was modified to introduce silent mutations to internal T₅NT motifs that encode early transcription termination signals for vaccinia virus. Elimination of the T₅NT sequences is known to minimize premature transcription termination and optimize foreign gene expression in vaccinia virus.

Full length gag gene encoding the p55 poly-protein was subcloned without any modifications.

The rev gene was modified in several ways. Twelve codons, encoding amino acids 75-86, were deleted and replaced with two codons, encoding aspartic acid and leucine, to render the rev protein non-functional. In addition, the nucleotide sequence of the rev gene was altered at codon position 3 (“wobbled”) to minimize homology between the tat and rev genes and to optimize expression of rev protein in human cells, “humanize” expression, without otherwise changing the amino acid sequence.

The first exon of the tat gene was modified by in vitro mutagenesis to change two codons, at amino acids 26 and 32, from tyrosine to alanine, to render the protein nonfunctional while preserving the 3-dimensional structure. In addition, the second exon of the tat gene was deleted. A comparable tat mutant was tested by the manufacturer in a transactivation assay and was unable to activate transcription of HIV-I LTR.

The modified tat and rev sequences were cloned as a fusion gene, with appropriate initiation and termination codons.

The nef gene was modified by changing codons at amino acids 62-65 from glutamic acid to alanine to reduce MHC class I downregulation and CD3 signaling.

The reverse transcriptase (RT) portion of the pol gene was modified by changing codons at amino acids 336 and 337 from aspartic acid to aspargine to eliminate reverse transcriptase activity. Protease and integrase sequences were not included in the construct.

The modified nef and RT coding sequences were fused in frame to form a nef-RT fusion gene. A calorimetric immunoassay was used to assess nef-RT for retroviral activity of a comparable construct; no enzymatic activity was detected.

TABLE 1
HIV-1C vaccine plasmid vector construct summary

| | env | gag | rev | tat | RT | nef |
|---|---|---|---|---|---|---|
| GenBank | AF067158 | AF067157 | AF067154 | AF067157 | AF067158 | AF067154 |
| Accession # | | | | | | |
| Gene | Full length, | Structural | Synthetic gene. | Exon 2 | Does not | Amino acids |
| identity in | InternalT5NT | protein | Position 3 | deleted. | include | 62-65 changed |
| construct + | removed to | only. | wobbled to | Two point | Protease | from 5E to 5A |
| modification | avoid | Pol | minimize | mutations | and | to reduce MHC |
| | premature | sequences | homology with | introduced | Integrase. | downregulation. |
| | transcription | not | tat, and to codon | to render | Amino | |
| | termination. | included. | optimize the rev | protein | acids 336 | |
| | | | sequence for | non- | and 337 | |
| | | | expression in | functional: | changed | |
| | | | human cells. | amino | from DD | |
| | | | A mutation is | acids 26 | to NN to | |
| | | | introduced at | and 32 | eliminate | |
| | | | position 75 | altered (Y | RT | |
| | | | (LLPLERLHISGS | to A). | activity. | |
| | | | to LE) to render | | | |
| | | | the protein non- | | | |
| | | | functional. | | | |
| Base (nt) | 2529 | 1473 | 291 | 216 | 1686 | 621 |
| Amino Acid | 843 | 491 | 97 | 72 | 562 | 207 |
| Protein size | 95 | 55 | 10.7 | 8.3 | 64 | 23 |
| (KD) | | | | | | |

| | | | | |
|---|---|---|---|---|
| Fusion | None | None | tat.rev | nef.RT |
| genes and | 95 | 55 | 19 | 85 |
| protein size | | | | |
| (KD) | | | | |

The DNA sequence of the transgenes (HIV IC env, gag, nef-RT and tat-rev) and associated transcriptional control regions that comprise TBC-M4 and about 800-900 bp of genomic viral sequences were determined. Two sequences were determined: the first includes the 49/50 region, the transgenes tat-rev and nef-RG and is designated TB19a.1. The second sequence, TB19a.2, contains the del III region and the transgenes env and gag contains the coordinates of features in the TBC-M4 insert. The determined sequences of the virus insert, 19a.1 and 19a.2, were aligned to the predicted TBC-M4 sequence.

TABLE 2
Position of features in TBC-M4 sequence.

| | | |
|---|---|---|
| Feature | Description | Position |

TB19a.1
49/50 insertion region

| | | |
|---|---|---|
| 5′ Virus sequence | Sequences outside of the insertion | 1 to 432 |
| | site | |
| 49/50 flanker | Insertion site | 433 to 971 |
| Tat-rev | Coding sequence | 995 to 1504 |
| 7.5K | Transcriptional control unit | 1556 to 1806 |
| nef-RT | Coding sequence | 1855-4164 |
| sE/L | Transcriptional control unit | 4208 to 4247 |
| 49/50 | Insertion site | 4278 to 4790 |
| 3′ Virus sequence | Sequences outside of the insertion | 4791 to 5252 |
| | site | |

TB19a.2
del III insertion region

| | | |
|---|---|---|
| 5′ Virus sequence | Sequences outside of the insertion | 1 to 501 |
| | site | |
| del III fl1 | Insertion site | 502 to 1428 |
| sE/L | Transcriptional control unit | 1434 to 1473 |
| env | Coding sequence | 1544 to 4075 |
| 40K | Transcriptional control unit | 4134 to 4292 |
| gag | Coding sequence | 4344 to 5819 |
| del III fl2 | Insertion site | 5837 to 6358 |
| 3′ Virus sequence | Sequences outside of the insertion | 6359 to 6815 |
| | site | |

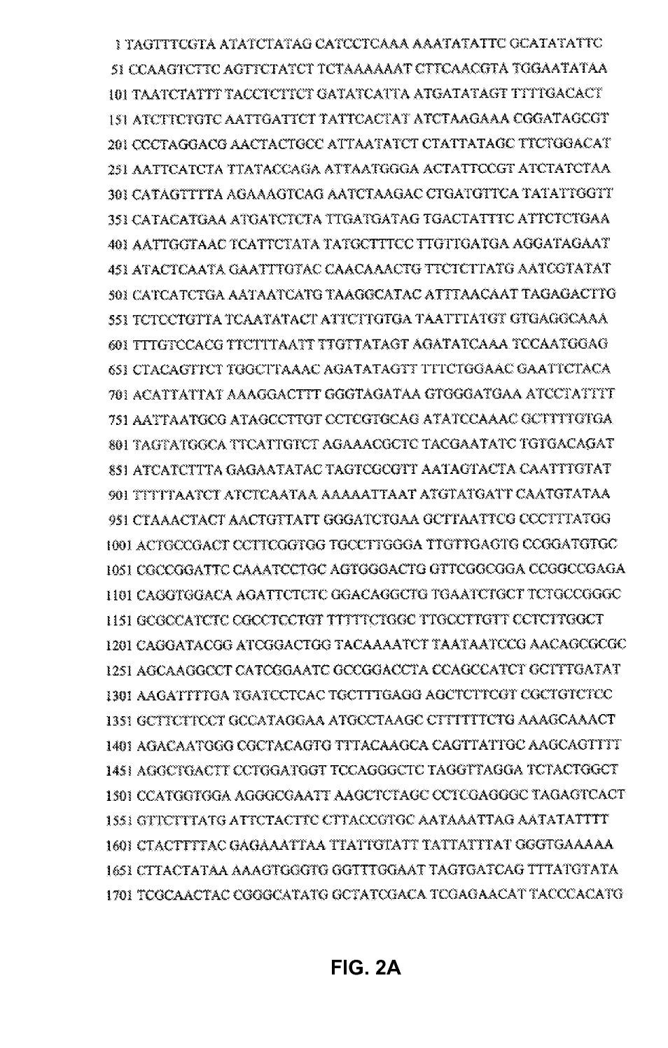

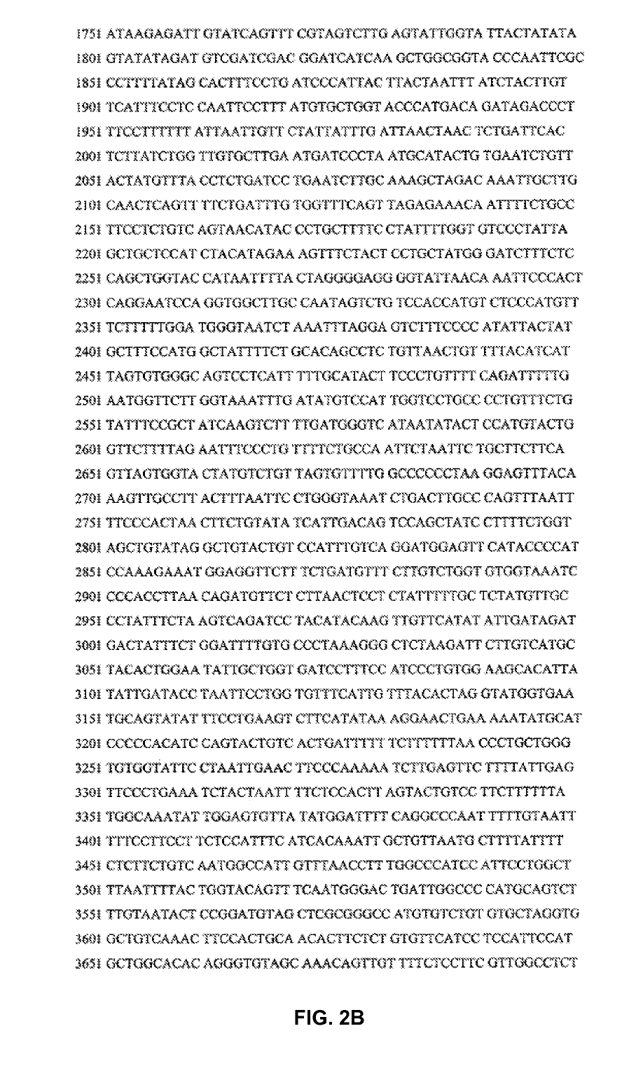

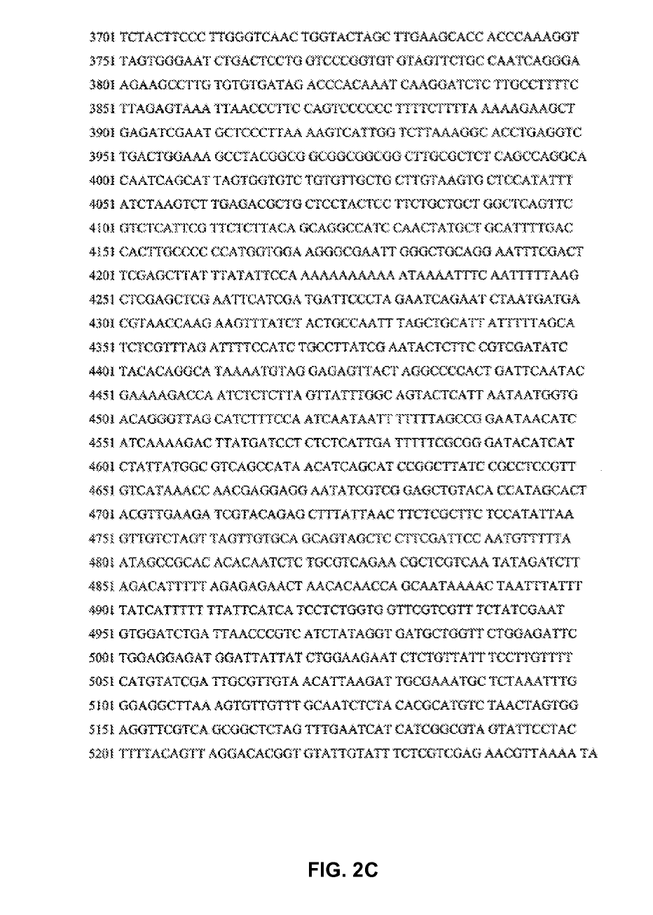

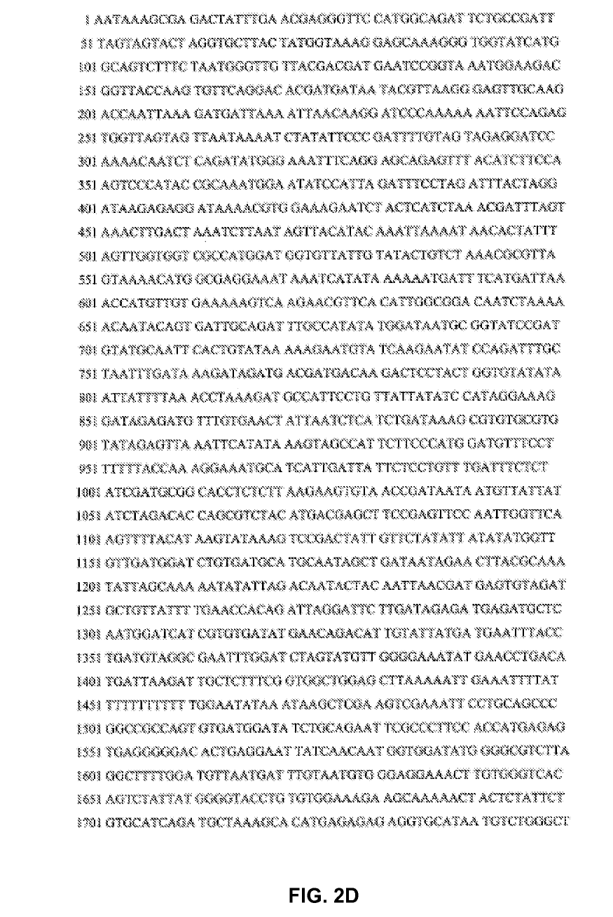

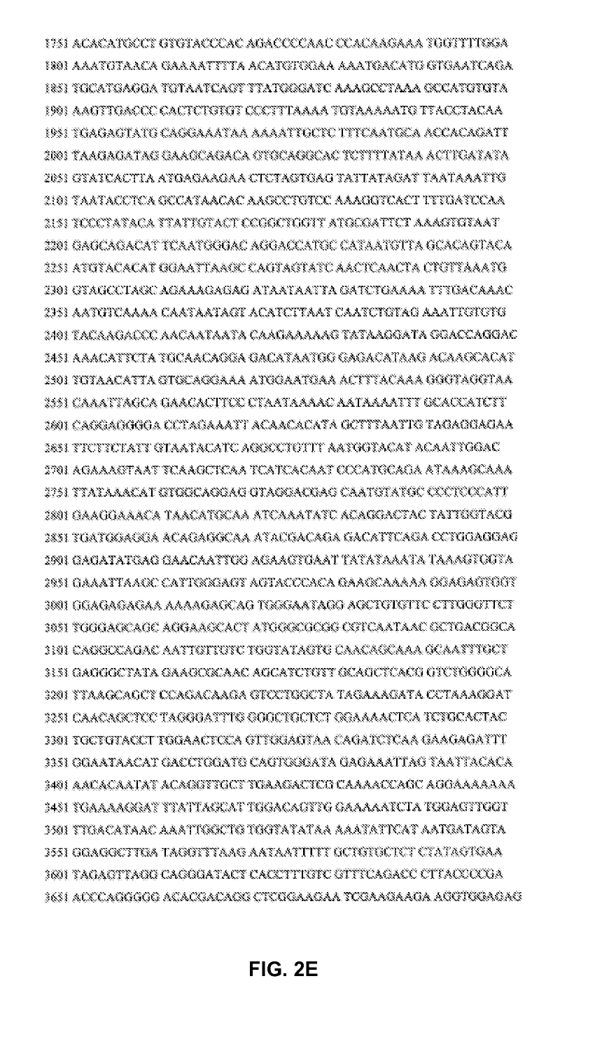

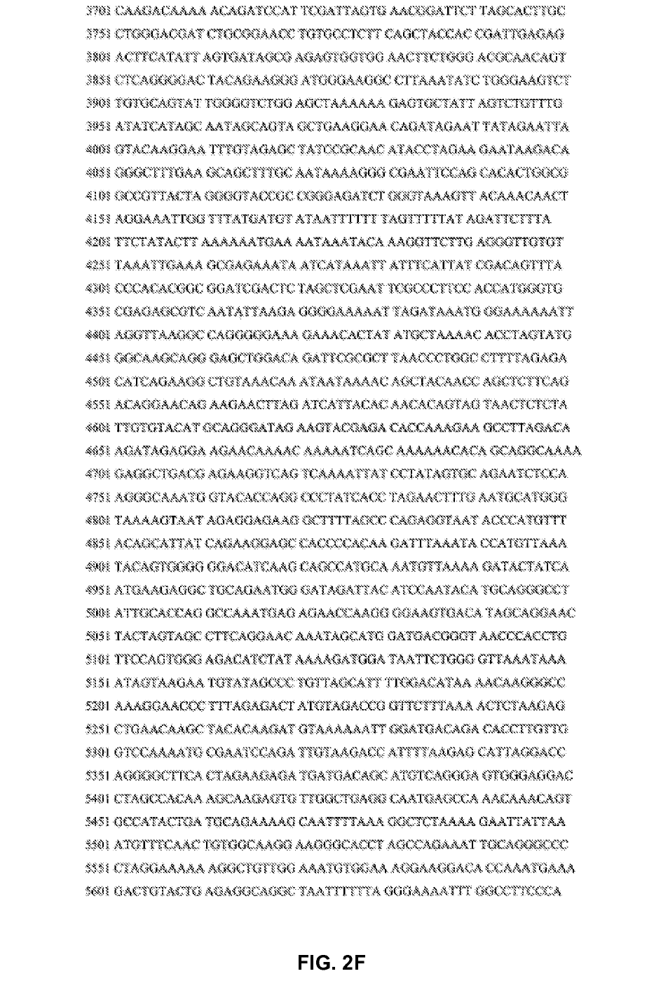

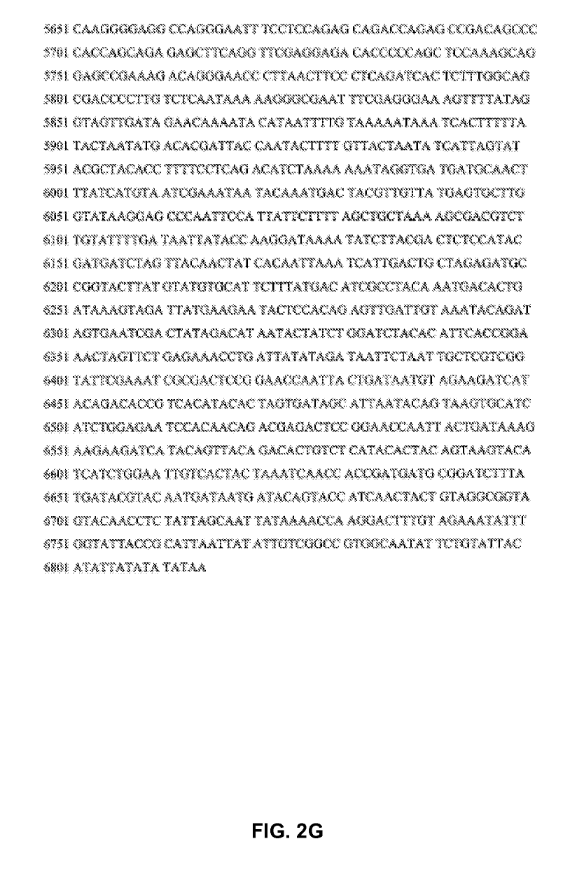

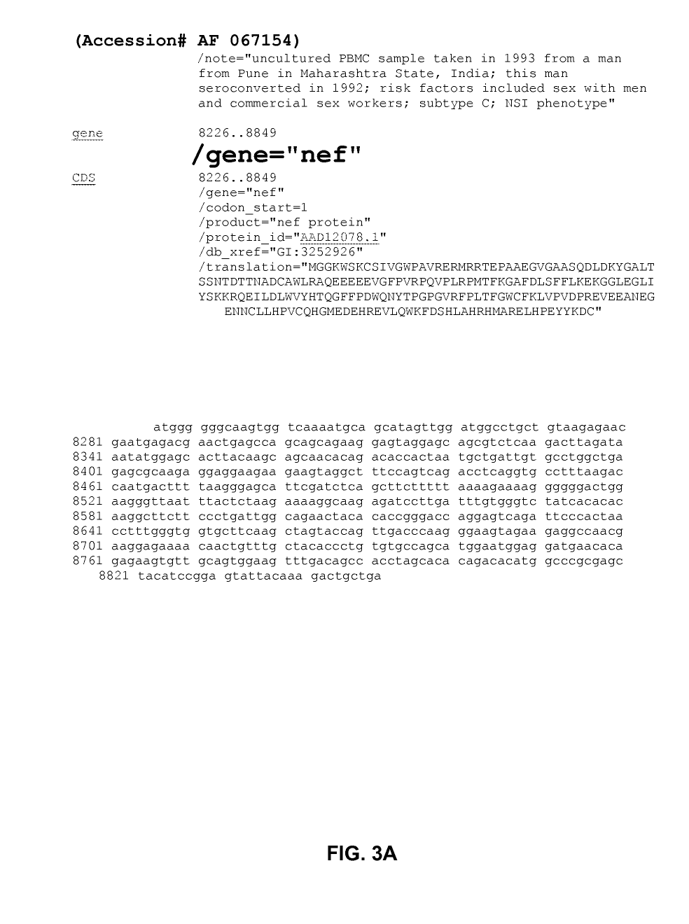

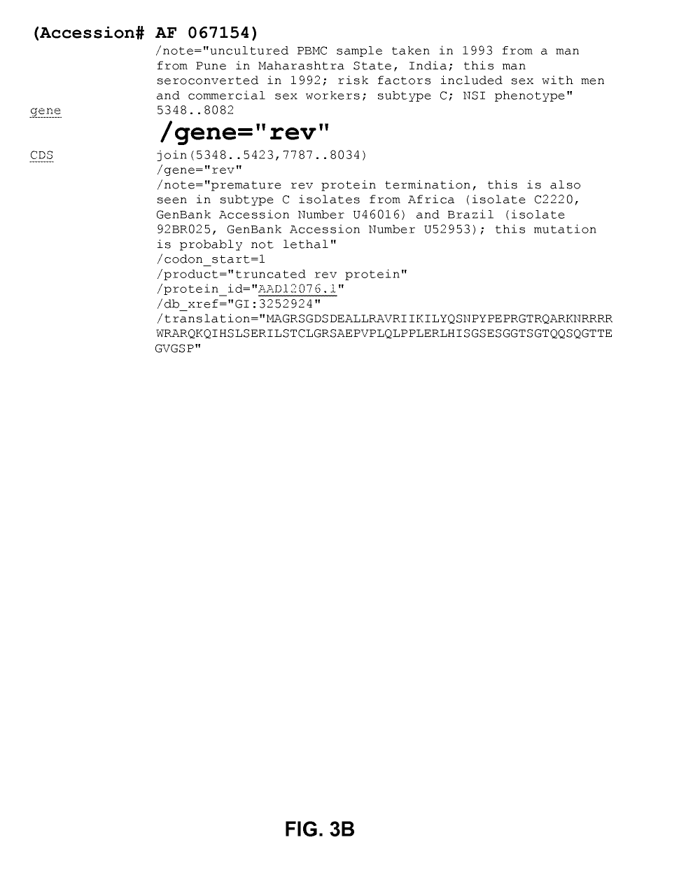

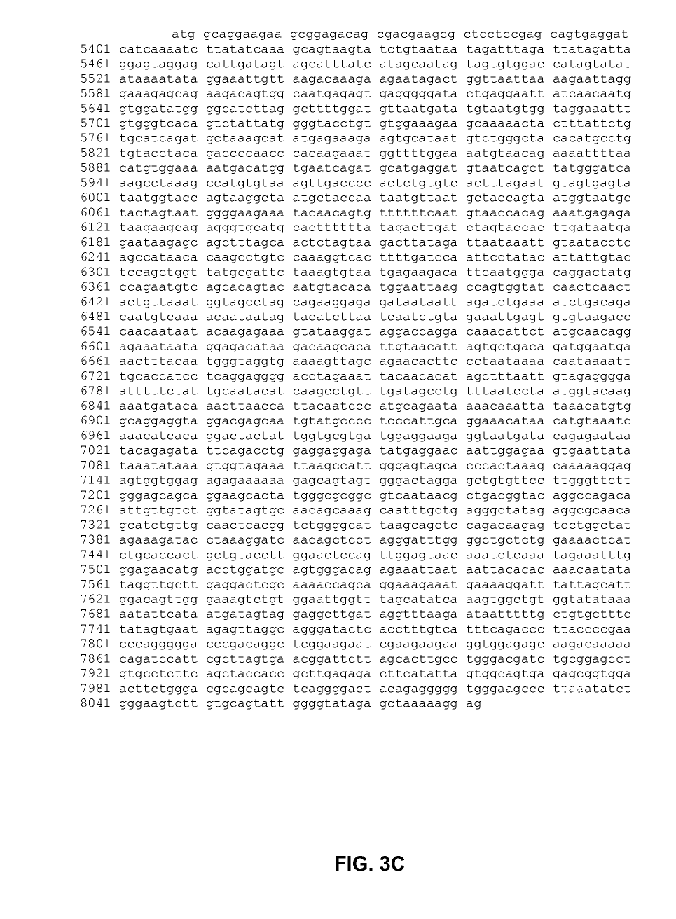

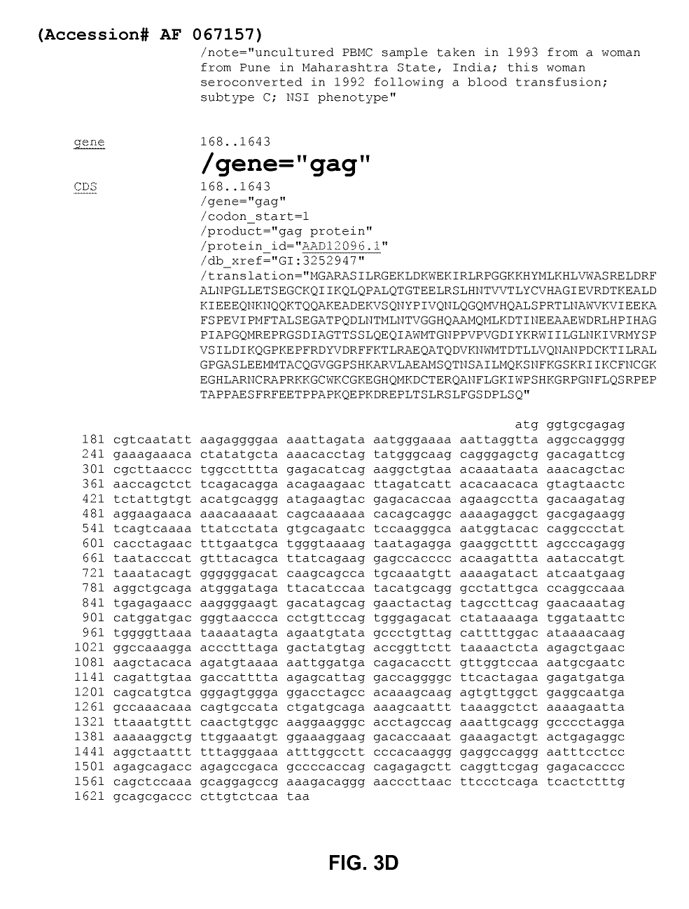

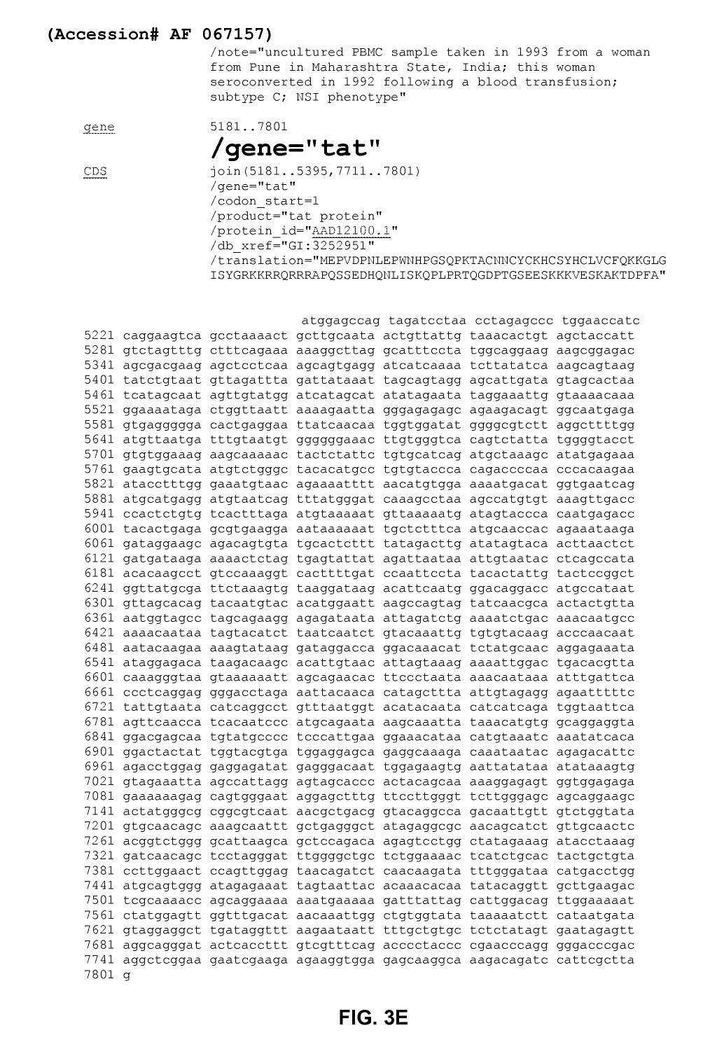

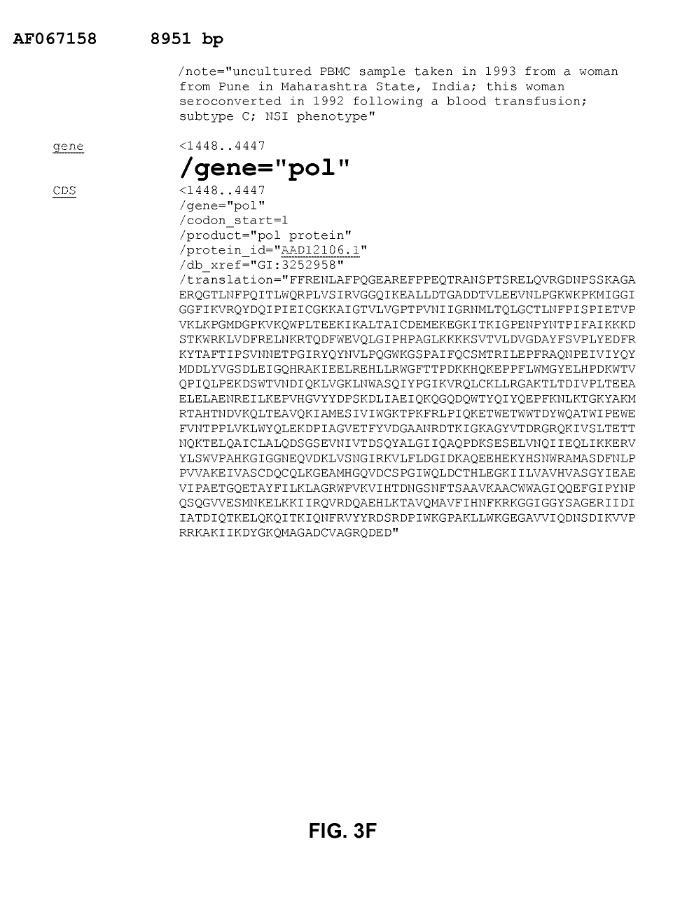

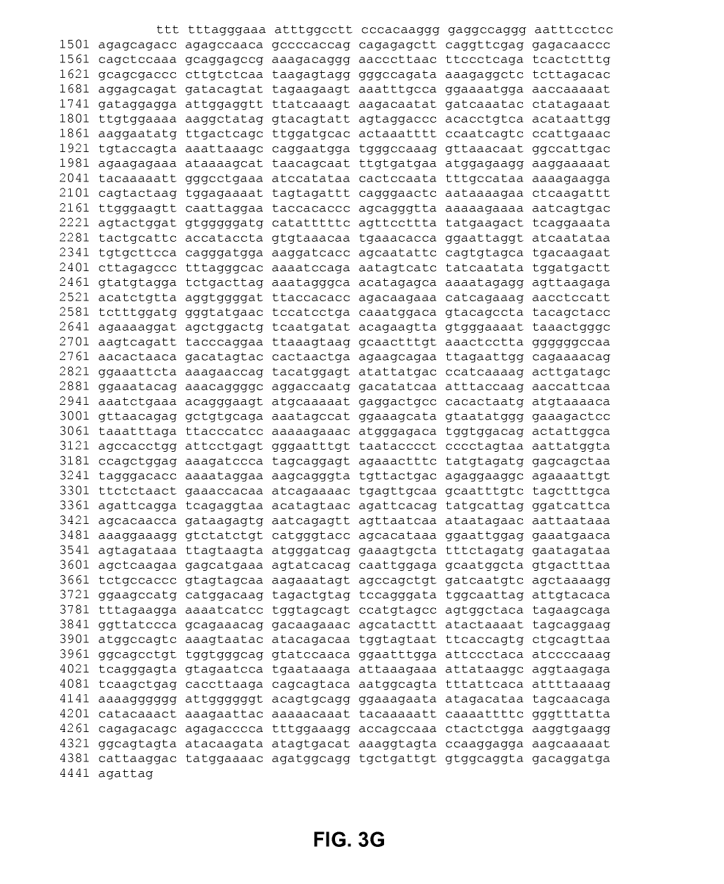

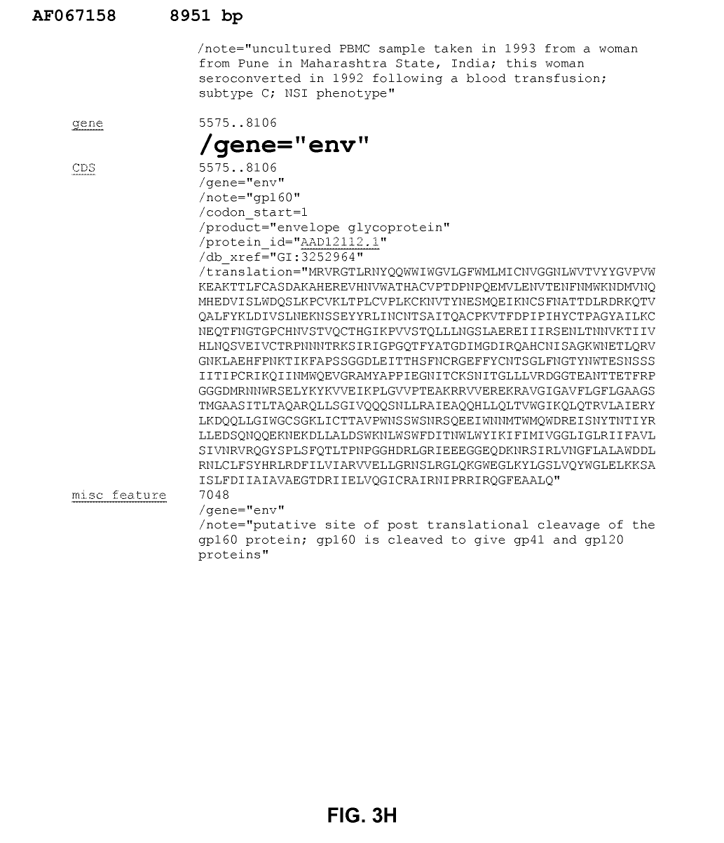

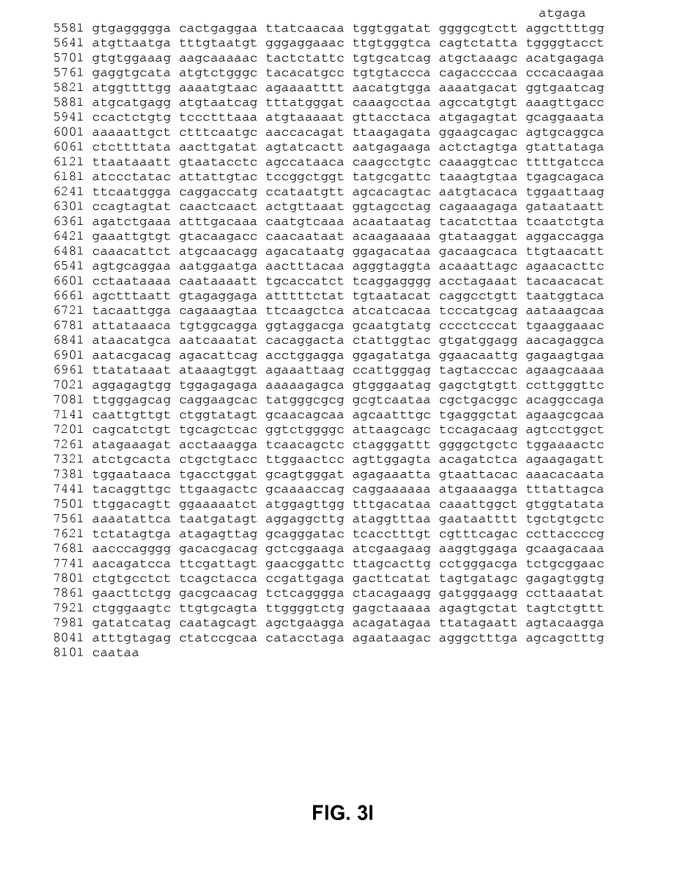

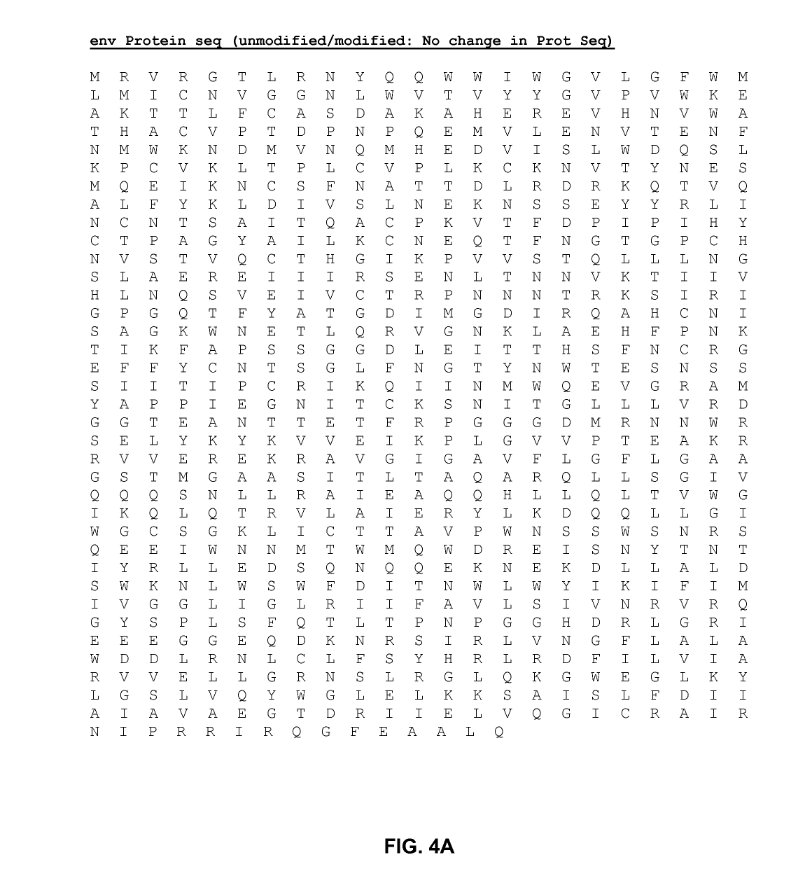

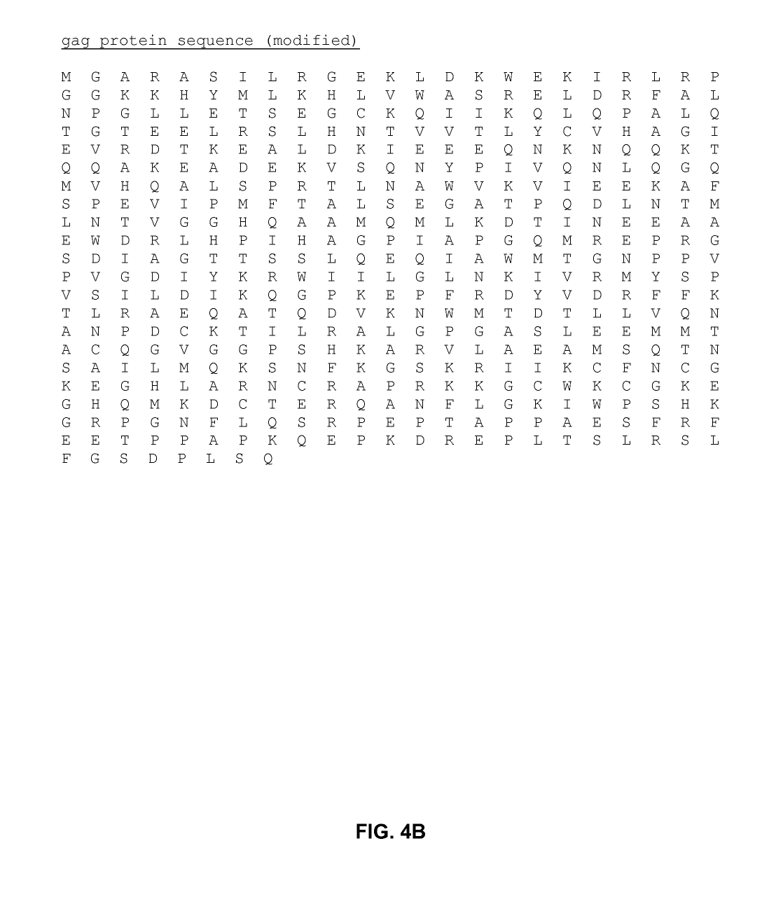

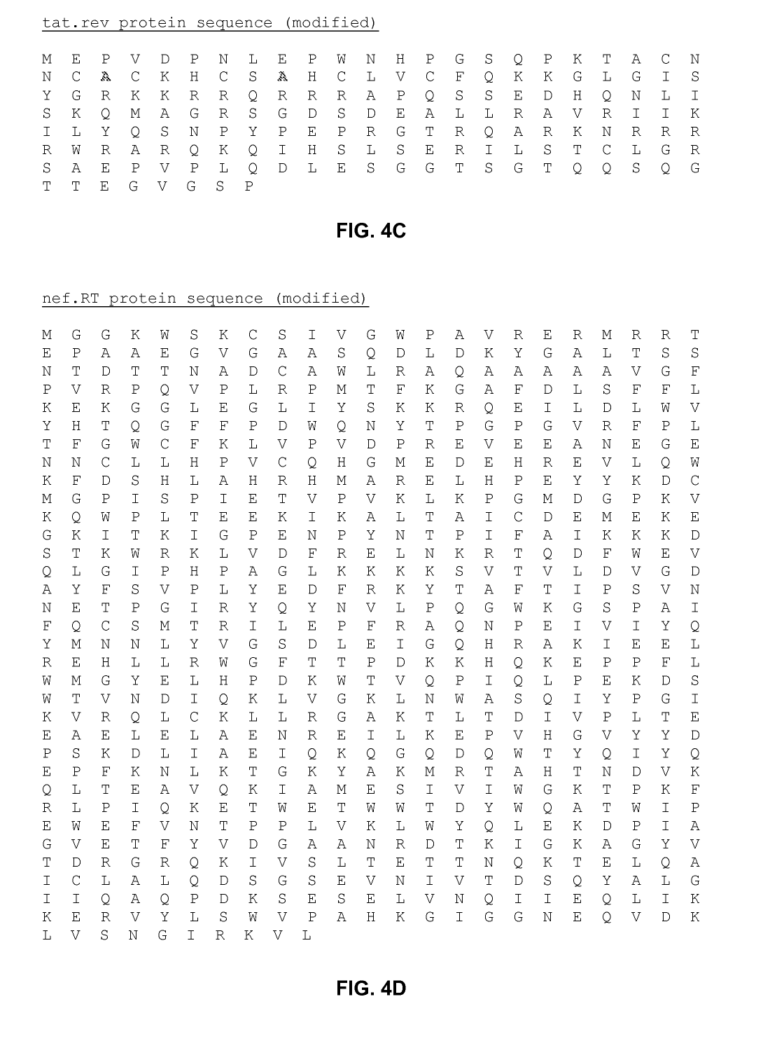

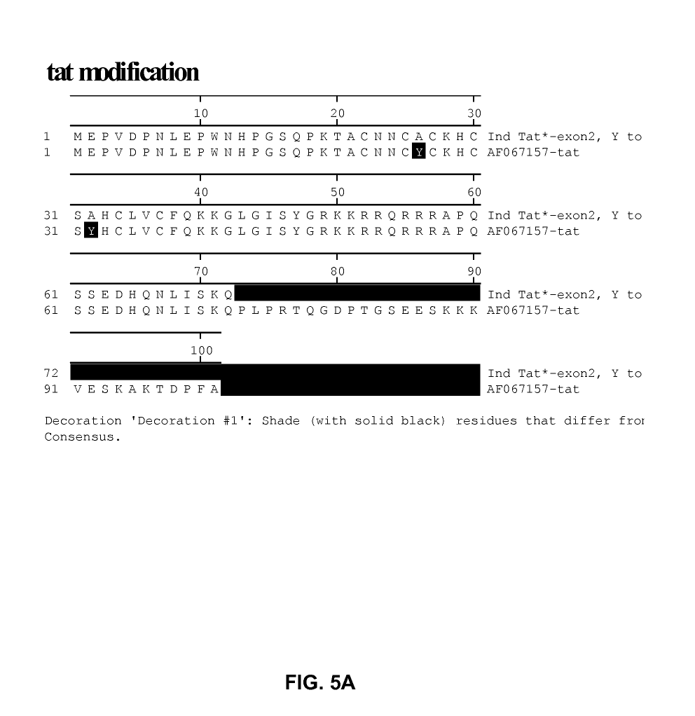

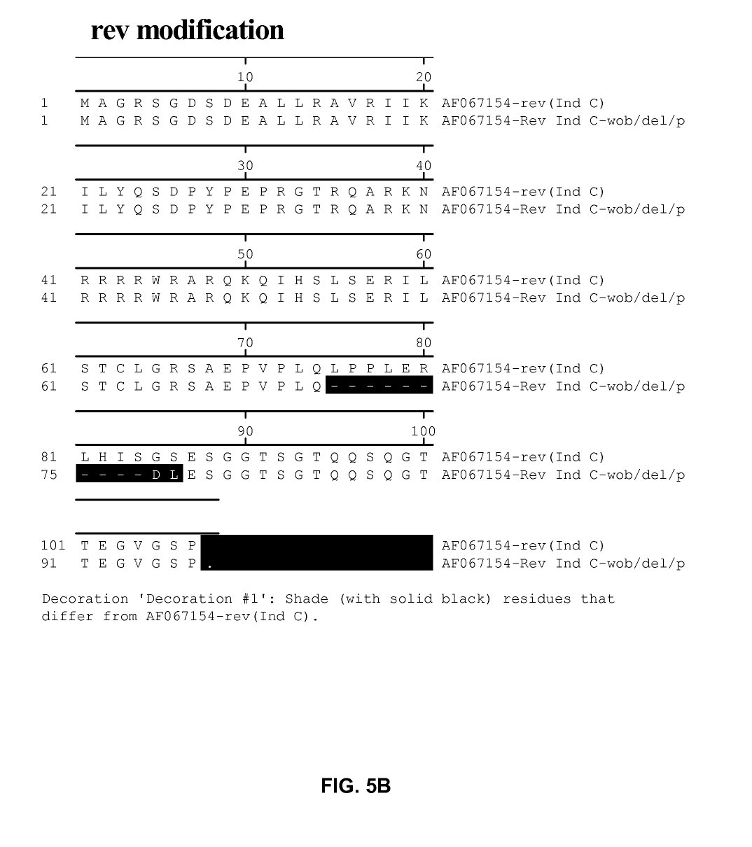

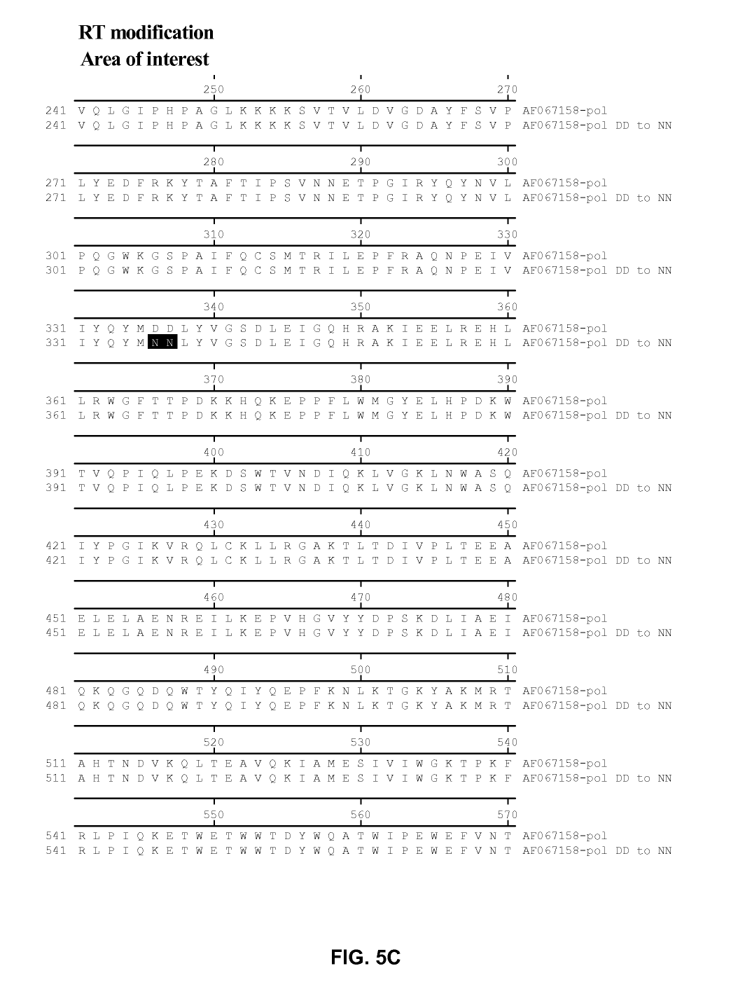

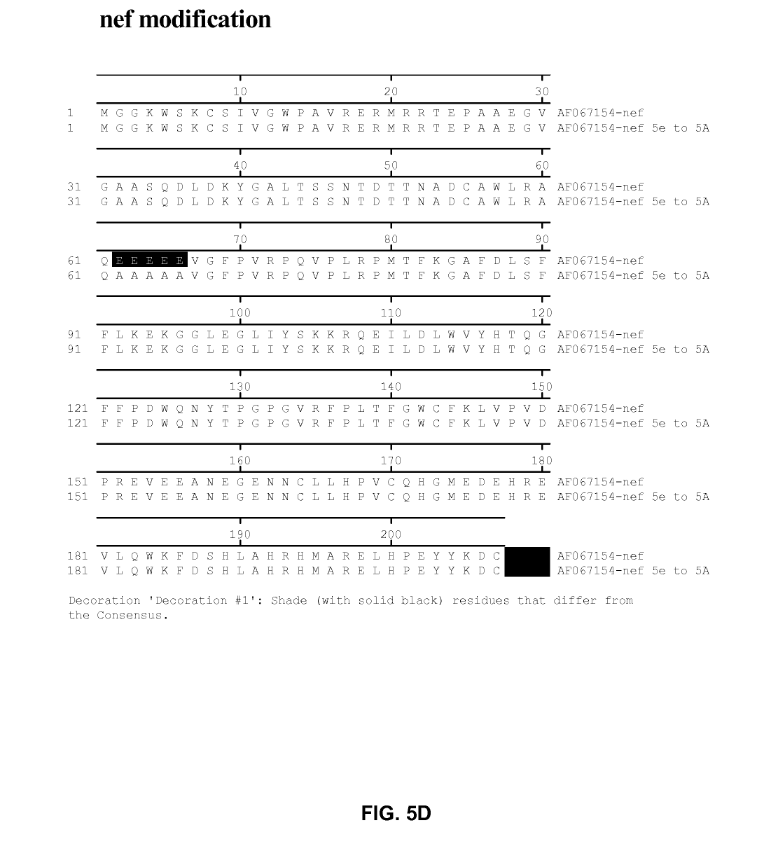

The sequences of the inserts are presented in FIGS. 2A-5D.

### Example 2

### Construction of the MVA Recombinant

The generation of recombinant MVA viruses is accomplished via homologous recombination in vitro between MVA genomic DNA and a plasmid vector that carries the heterologous sequences to be inserted. The plasmid vector contains the foreign sequences flanked by viral sequences from a non-essential region of the MVA virus genome. The plasmid is transfected into cells infected with the parental MVA virus, and recombination between MVA sequences on the plasmid and the corresponding DNA in the viral genome results in the insertion into the viral genome of the foreign genes on the plasmid.

The plasmid vector that was constructed contained the following elements (1) a prokaryotic origin of replication to allow amplification of the vector in a bacterial host; (2) the gene encoding resistance to the antibiotic ampicillin, to permit selection of prokaryotic host cells that contain the plasmid; (3) DNA sequences homologous to the deletion III region of the MVA genome, that direct insertion of foreign sequences into this region via homologous recombination; and (4) a set of chimeric genes, each comprising a poxyiral promoter linked to an HIV-1 gene.

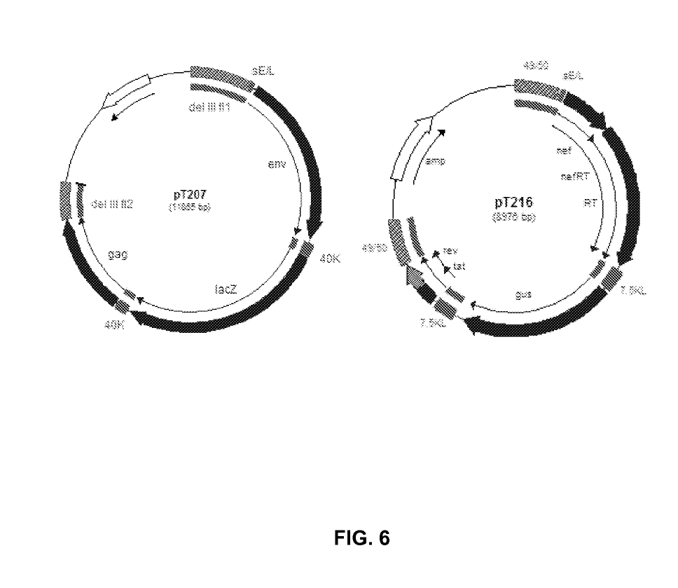

FIG. 6 depicts an annotated plasmid map of a transfer vector. The size of the transfer vector is 177923 bp and functional components include amp gene, poxvirus promoters—sE/L, 40K and 7.5K, MVA insertion sites—del III and 49/50, reporter genes—lacZ and gus and HIV-1C antigens (env, gag, tat-rev and nef-RT).

In the human clinical trials, live recombinant pox viruses have proven to be well tolerated and immunogenic, eliciting both antibody and cell-mediated immune responses. MVA has the advantage of not replicating in human cells and has proven safety record in over 120,000 vaccinated individuals. In addition, MVA DNA replication and gene expression are relatively unimpaired in human cells, allowing high level of expression of foreign proteins, which may result in more potent immune responses upon vaccination. MVA has good safety record and can induce both antibody and cell-mediated immune response, including antigen-specific MHC-class I restricted CTLs.

MVA originated from the Dermovaccinia strain CVA. CVA was retained for many years at AVS (Ankara Vaccination Station) via donkey-calf-donkey passages. In 1953, the virus was purified and passaged twice through cattle. In 1954/55 CVA was used in the Federal Republic of Germany as a smallpox vaccine. In 1958, attenuation experiments by terminal dilution of CVA was begun in chicken embryo fibroblasts (CEF). After 360 passages, the virus was plaque purified three successive times and subsequently replicated in CEF until passage 570 was achieved. The virus was once again plaque purified on CEF prepared from a recognized avian leukosis virus-free flock of chickens. Two vials of lyophilized original seed virus labeled “MVA” Saatvirus 575. FHE-K. v.14.12.83 (translation: MVA Seed virus, passage 575, Chicken Embryo Fibroblasts-K from Dec. 14, 1983) were received and lyophilized virus was kept unopened at 4° C. until it was used.

The starting material for the production of TBC-MVA was one of the MVA Saatvirus 575. FHE-K. v.14.12.83 vials obtained in 1995. One vial of the original seed virus was reconstituted with 1 mM Tris pH 9.0, aliquotted and then serially diluted in DME supplemented with 0.1% FBS (DME/0.1% FBS) in preparation for plaque purification on primary chicken embryo dermal (CED) cells. The diluted virus was passaged in CED cells to produce the TBC-MVA seed stock lot #1-9.

Twenty 850 cm² roller bottles were seeded at 6×10⁷ CED cells/roller bottle and infected with TBC-MVA Seed Stock Lot #1-9 at an MOI of 0.1 pfu/CED cell. The roller bottles were then sparged with 10% CO₂/20% O₂/balance N₂ and placed on roller racks in the warm room. Infection was allowed to proceed for 4±1 days at 34.5±1.5° C. At the end of the infection period, infected cells and culture medium were harvested and samples generated for in-process testing (Crude Bulk). The infected cell suspension was centrifuged at low speed, the supernatant discarded and the pelleted cells resuspended in 1 mM Tris, pH 9.0. The pelleted cells were centrifuged at low speed, and the supernatant was harvested (Clarified Bulk). The pellet was resuspended in 1 mM Tris, pH 9.0 and the suspension was again centrifuged at low speed. The resulting supernatant was added to the Clarified Bulk. A sample was removed for titration and the Clarified Bulk was aliquotted into cryovials which were stored at −70° C. or colder. The master virus stock was designated TBC-MVA MVS Lot # 1-030599.

This TBC-MVA MVS Lot # 1-030599 (diluted) 1×10⁷ May 16, 2001 was used as parent virus to generate TBC-M420 (Indian HIV-1C env, gag, tat-rev, nef-RT) recombinant.

The TBC-M420 recombinant virus was generated using standard techniques of in vivo recombination. CED cells were infected with the parental MVA virus (TBC-MVA master virus stock). Using the calcium phosphate precipitation method, cells were then transfected with the plasmid transfer vector pT207 and pT216. After 48 hours, infected cells were harvested and progeny virus was released by three rounds of freezing and thawing.

Recombinant progeny viruses were identified using a chromogenic assay, performed on viral plaques in situ, that detects expression of the lacZ and gus gene product. Viral progeny obtained after in vivo recombination were used to infect monolayers of CED cells in 6 cm tissue culture plates. Approximately 24 hours later, an agarose solution was laid over the infected cells. Four days after the initial infection, an agarose solution containing the histochemical substrate Bluo-Gal/Magenta was applied. The Bluo-Gal/Magenta were converted by the products of the lacZ gene and gus gene, producing a purple precipitate in those plaques expressing these enzymes. The next day, positive plaques, which appeared purple against a light red background, were picked using sterile pasteur pipettes. These plaques were subjected to additional rounds of purification, until a pure plaque isolate was obtained.

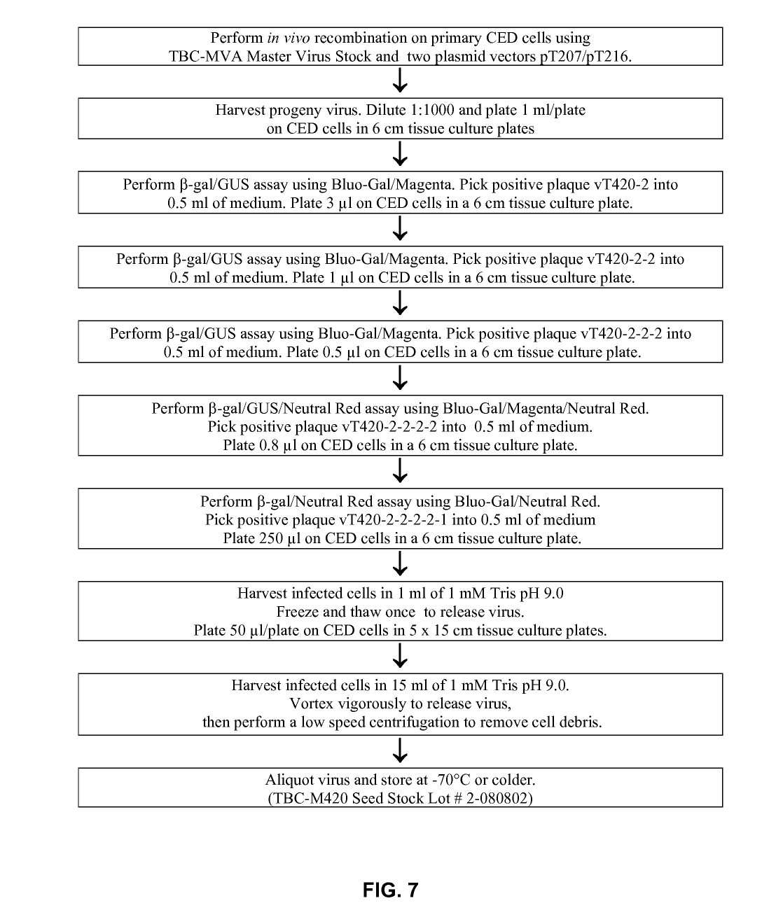

A flow chart outlining the isolation of the TBC-M420 recombinant and the preparation of the seed stock is shown in FIG. 7. To prepare the seed stock, the virus present in this final plaque pick underwent two rounds of amplification, the first in one 6 cm tissue culture plate, and the second in ten 15 cm tissue culture plates. The infected cells were harvested and progeny virus was released by three rounds of freezing and thawing. The virus was then aliquotted into cryovials and stored at −70° C. or colder. This stock, designated TBC-M420 Seed Stock Lot # 2-080802, serves as the starting material for the preparation of the recombinant master virus stock for vaccine production.

For genomic analysis of TBC-M420, the test Article was TBC-M420 SS Lot #2-080802, the negative control was TBC-MVA Lot # 1-030599 and positive controls were pT207 Lot # 01-060502 and pT216 Lot # 01-060502.

Test article genomic DNA was prepared by infecting chicken embryo dermal cells with TBC-M420 and extracting MVA genomic DNA. The DNA was analyzed by restriction endonuclease digestion with BamH I, EcoR I and Xba I; each restriction endonuclease digestion was performed with a single enzyme. The products of digestion were then separated by agarose gel electrophoresis and stained using ethidium bromide to visualize the DNA fragments. DNA fragments were transferred to nylon membranes for Southern blot hybridization. Each digest was probed individually with digoxigenin-labeled DNA corresponding to env, gag, del III, tat-rev, nef-RT and 49/50 sequences. As positive controls, the analysis was performed using plasmid pT207 Lot # 01-060502 for env, gag and del III; plasmid pT216 Lot # 01-060502 for tat-rev, nef-RT and 49/50. As a negative control, the analysis was performed using DNA prepared from non-recombinant MVA virus, TBC-MVA Lot # 1-030599. The sizes of the hybridizing fragments were compared to their expected sizes to determine whether fragments of the appropriate molecular weights contain the probe sequences. All of the predicted fragments were observed.

Non-expressors for the env gene were observed in the seed stocks.

TABLE 3
Stability of env expression by plaque assay

| | % non-expressor | | | |
|---|---|---|---|---|
| | | Passage #1 | Passage #2 | |
| Seed Stock Lot | Seed Stock | (MVS) | (MVS/Pd) | Passage #3 |
| 1 | 2.1 | 3.0 | 6.7 | — |
| 2 | 1.4% | 2.9% | 5.3% | 9.6% |
| | | (MOI 0.1) | (MOI 0.1) | (MOI 1) |
| | | | 3.7 | 8.4% |
| | | | (MOI 1) | (MOI 1) |
| | | | | 10.6% |
| | | | | (MOI 0.1) |

Western blot analysis revealed all genes were expressed (data not shown). TBC-M420 seed stock #2-080802 is an MVA recombinant encoding for the HIV-1 clade C ENV, GAG, TAT-REV and NEF-RT fusion proteins. The expression of these genes/proteins was determined by western blot analysis. In brief, recombinant infected cell lysates/proteins were separated by SDS-PAGE and transblotted onto nitrocellulose membrane paper. These blots were incubated with antibodies specific for the detection of HIV-1 ENV(gp120), GAG, REV, TAT, NEF, and RT. They were subsequently developed with a chromogenic substrate. Bands of the characteristic sizes (ENV=160/120 kD; GAG=55 kD, TAT-REV=29 kD and NEF-RT=90 kD) are considered to be positive evidence of gene expression.

A MOI of 2 was used due to the low titer of the test article and its limited availability, TBC-M420 SS #2-080802. All other recombinants were adjusted to the lowest titer for consistency. In all the blots, the band intensity for the TBC-M420 SS #2-080802 was stronger than the positive control, TBC-M395 SS #1-121801, due to the fact that the genes for the TBC-M420 SS #2-080802 are under a stronger promoter than TBC-M395 SS #1-121801.

Envelope:

TBC-M420 SS #2 was positive for bands of 160 and 120 kD sizes. The positive control TBC-M395 SS#1 was positive for a band of 160 and 120 kD. However, this is not that detectable on the scans, the original blot does show the appropriate band. The negative control, TBC-MVA did not have these bands present, confirming that the conditions were specific for the detection of HIV-1 Envelope.

Gag:

TBC-M420 SS #2 was positive for bands of 55/45 kD sizes. The positive control TBC-M395 SS#1 was positive for a band of 55/45 kD. The negative control, TBC-MVA did not have these bands present, confirming that the conditions were specific for the detection of HIV-1 GAG.

Nef and RT:

TBC-M420 SS #2 was positive for bands of 90 kD sizes. The positive control TBC-M395 SS#1 was positive for a band of 90 kD. However, the positive control scan, TBC-M395 SS#1, does not show a prominent band at 90 kD. On the original blot, the band is detectable. The fact that bands of the same sizes were detected under both antibody conditions confirms that the gene expressed is a single polyprotein. The negative control, TBC-MVA did not have these bands present, confirming that the conditions were specific for the detection of HIV-1 NEF and RT.

TAT and REV:

TBC-M420 SS #2 was positive for bands of 29 kD sizes. The positive control TBC-M395 SS#1 was positive for a band of 90 kD. The fact that bands of the same sizes were detected under both antibody conditions confirms that the gene expressed is a single polyprotein. The negative control, TBC-MVA did not have these bands present, confirming that the conditions were specific for the detection of HIV-1 TAT and REV.

As noted previously, purity of expression of genes other than env (plaque analysis) has not been performed due to lack of a suitable assay.

Titration of the virus was performed using primary CED cells in 6 cm tissue culture plates. The virus was serially diluted in culture medium and the dilutions were applied to the cells. Approximately 24 hours after infection, the culture medium was removed and an agarose overlay was applied to the infected cell monolayer. Three days later, a second agarose overlay containing neutral red was applied. After an additional two-day incubation, the total number of plaques on each plate was counted and the titer in plaque-forming units (pfu)/ml was calculated using counts from plates containing 20-200 plaques. The concentration of the TBC-M420 Seed Stock Lot # 2-080802 was determined to be 8.8×10⁷ pfu/ml.

The AIDS Vaccine Evaluation Groups (AVEG) have conducted a number of Phase I clinical trial protocols to evaluate pox virus-based AIDS vaccine candidates. Protocols 002, 002A, 002B, 008, and 010 have tested a prime-boost regime using a replicating vaccinia virus that expresses an HIV-1 env gene (HIVAC-1e) in combination with a variety of HIV env subunit preparations. Similarly, protocols 014A and 014C have evaluated Therion's multigenic recombinant TBC-3B, which expresses env and gag-pol genes from a 3β isolate of HIV-1. In 014C, TBC-3B-immunized volunteers were boosted with an HIV env preparation. The remaining trials utilized various canarypox recombinants (generated by Pasteur Merieux Connaught) expressing one or more HIV genes, in combination with a variety of different subunit boosts. Thus, there is ample experience with the use of replicating and non-replicating pox virus-based vaccines in clinical trials.

In these human clinical trials, live recombinant vaccinia virus has proven to be well tolerated and immunogenic. Similarly, the canarypox recombinants were well tolerated and elicited both antibody and cell-mediated immune responses; however, some concern has been raised regarding the potency of the immune responses elicited by the canary pox recombinants, with recent data indicating that only about half of all vaccines develop even transient HIV-specific CTL responses.

MVA recombinants may combine the best features of avipox and replicating vaccinia viruses. The vector's inability to replicate in human cells and proven safety record in over 120,000 vaccinated individuals address concerns raised by the use of replication-competent vaccinia. However, in contrast to avipox, MVA DNA replication and gene expression are relatively unimpaired in human cells; this feature, which allows high level expression of foreign proteins, may result in more potent immune responses upon vaccination.

### Example 3

### Animal Data

The intended pharmacological effect of the TBC-M4 vaccine is the induction of an immune response to the target HIV-1 proteins that have been inserted into the Modified Vaccinia Virus (MVA) viral vector. All six selected HIV-1 proteins: env, gag, nef, RT, tat and rev, have been shown to be expressed by the recombinant MVA virus as assessed by Western blot (Example 2). The objective of the preclinical pharmacology studies was to assess the biological activity of the vaccine in vivo. Assessment of host immune responses to the viral vector, MVA, and the encoded HIV-1 proteins were used to assess biologic activity of the TBC-M4 vaccine candidate.

The proposed mechanism of action for the TBC-M4 vaccine is that the recombinant MVA virus will infect human cells, undergo limited replication and in turn the cells will express the inserted HIV proteins. The expression of the HIV-1 antigens in the human subjects exposed to TBC-M4 vaccine should elicit host cellular and humoral immune responses. It is hypothesized that the elicited broad range immune responses to the env, gag, nef, RT, tat and/or rev proteins may significantly reduce viral exposure and sequella in the host upon subsequent exposure to the human immunodeficiency virus (HIV). Supporting information on the proposed mechanism of action is provided below.

Studies of HIV infection in humans and SIV Infection in rhesus monkeys have demonstrated an important role for neutralizing antibodies. Targeted insertion of HIV genes into live attenuated viruses that induce potent humoral and cellular responses is considered a feasible strategy for induction of protective Immune responses against HIV.

Modified Vaccinia Ankara virus is a live attenuated strain derived from wild type vaccinia virus by serial passage through chick embryo fibroblast (CEF) cells. During the attenuation process, MVA virus underwent multiple well-characterized genomic deletions that have been associated with its reduced pathogenicity. The genomic deletions have been extensively characterized and appear to affect late stage virion assembly and expression of cytokine receptors. As a consequence, the modified virus is able to infect most mammalian (including human) cells and to express viral (and recombinant) genes in the normal way, but does not replicate efficiently in most primary cell types or immortalized cell lines After two decades of study, productive replication of MVA virus is largely considered to be restricted to chicken embryo fibroblast cells.

Unlike the CVA parental strain, MVA virus does not express soluble receptors for a range of cytokines including IFN-γ, IFN-αβ, TNF and chemokines; it does, however, express a soluble IL-1β receptor and has proven to be a potent inducer of humoral immune responses, Type I IFN, and CD8⁺ cells in a variety of disease models.

The exact mechanisms by which the foreign genes inserted into MVA virus are expressed, and the relevant antigens presented so as to induce specific immunity, remain unclear. It is presumed that the six HIV-1 polypeptides will be processed and presented in the context of MHC Class I following expression In infected cells. Humoral responses may be elicited by the secretion of antigen from virus-infected cells, or by the release of such antigen following cell lysis. Antigen released by these means may then be taken up by professional antigen-presenting cells (APCs) and presented to CD4⁺ T-cells in the draining lymph nodes.

The mechanism of presentation of genetically introduced antigens to CD8⁺ responses by recombinant MVA virus is less well understood, but induction of these cells has been demonstrated in HIV, SIV, and other disease models. Animal studies have demonstrated the induction of specific CD8⁺ responses by recombinant MVA virus expressing HIV-1 subtype A or SIV CTL epitopes in both mice and rhesus monkeys. In the mouse, administration by the intravenous route gave a better response than by the intramuscular route while administration by intradermal injection was also effective. In the rhesus monkey, immunized animals showed lower viral load and prolonged survival following subsequent challenge compared with controls, although complete protection was not shown.

The intended pharmacologic effect of the TBC-M4 vaccine is the induction of cellular and humoral immune responses to the target HIV-1 proteins encoded by the MVA viral vector. All six selected HIV-1 proteins; env, gag, nef, RT, tat and rev have been shown to be expressed by the recombinant MVA virus as assessed by Western blot of primary CED cells and non-human primate and human cell lines. Immune responses to the vaccine have been assessed using an ELISA to measure vaccinia (MVA virus) binding antibodies and an enzyme-linked immunospot (ELISPOT) gamma interferon assay to measure cellular immune responses to the HIV-1 target gene products.

The ability of the TBC-M4 vaccine to induce host immunity has been independently verified in three animal models: rodents (mice), rabbits and non-human primates.

Two classes of immune responses, humoral and cellular, have been measured in animals exposed to the TBC-M4 vaccine. An ELISA method is utilized to detect vaccinia binding antibodies in sera. An ELISPOT interferon gamma assay is used to detect T-cell responses to the target HIV-1 antigens.

Anti-vaccinia humoral responses. For other recombinant poxvirus based vaccines in phase I clinical development, induction of vaccinia binding antibodies in sera of exposed animals has been utilized as the primary indicator of pharmacologic activity. The ELISA to measure vaccinia-binding antibodies has been validated for assay of human and mouse sera and qualified for rabbit sera. Measurement of vaccinia binding antibodies was initially performed to demonstrate immunogenic potential of the vaccine and in subsequent studies to verify pharmacologic activity of TBC-M4 vaccine in the two nonclinical toxicology studies.

HIV-1 specific ELISPOT gamma interferon assay. In preparation for later stage clinical development, assays and reagents are being developed to measure antigen specific T cell responses to the vaccine. An ELISPOT assay that detects splenic IFN-gamma producing cells has been developed to measure antigen specific cellular immune responses following in vitro stimulation. Two studies measuring antigen specific cellular immune responses have been conducted with the TBC-M4 vaccine.

Immunogenicity of TBC-M4 in mice. The objective of the study was to evaluate the anti-vaccinia, anti-gag, and anti-env humoral responses of mice following intramuscular exposure to TBC-M4. The ELISA methods used for assay of anti-env and gag immunoglobulin responses were developed with subtype B antigens and cross-reactivity with immunoglobulin raised against subtype C antigens was not established prior to assay of serum in from this study.

A clinical lot of TBC-M4 vaccine was in a frozen state at a stock concentration of 1×10⁸ pfu/ml. The test material and placebo (PBS/10% glycerol) were stored frozen until use. On each day of test article administration a new dosing solution was prepared by making a 1 to 10 dilution of the stock material in placebo to yield a 1×10⁷ pfu/ml working solution. Female BALB/c mice were selected as the animal model. On each dosing occasion the animals received 100 μl of test material delivered in two 50 μl intramuscular injections, one into each of the two hind limbs.

Blood was collected from each animal prior to SD 0 and two weeks following each dosing occasion. Pre- and post-immunization samples were assayed for serum vaccinia binding responses by ELISA. Reported titers were determined based on the OD value measured in naive sera times three. The limit of detection is a titer of 100, which indicates that at a 1:100 dilution the OD of the sample is comparable to negative control wells.

Data from the anti-vaccinia ELISA are provided in Table 4. Antivaccinia titers were detected in four of six mice within two weeks of the first vaccine administration, SD 14. Serum samples from animals vaccinated two or more times (SD 0 and 21 or SD 0, 21, and 35) were all positive (12 of 12) in the vaccinia antibody bindingELISA. The results obtained In the vaccinia immunoglobulin assay verified the pharmacologic activity of the vaccine in the mice and indicated that TBC.M4 vaccine begins to induce a detectable host immune response after primary exposure.

TABLE 4
Post-immunization antibody titers

| | | | Anti- | Anti-env | Anti-gag |
|---|---|---|---|---|---|
| | Serum | Test Article | vaccinia | (Clade B) | (Clade C) |
| Group | Collection | Admin | Titer 1/Dilution | | |
| A | Day 14 | Day 0 | 1600 | <100 | <100 |
| | | | 3200 | <100 | <100 |
| | | | 6400 | <100 | <100 |
| | | | 12800 | <100 | <100 |
| | | | <400 | <100 | <100 |
| | | | <100 | <100 | <100 |
| B | Day 35 | Day 0; Day | 1600 | <100 | <100 |
| | | 21 | 800 | <100 | <100 |
| | | | 1600 | <100 | <100 |
| | | | 12800 | <100 | <100 |
| | | | 800 | <100 | <100 |
| | | | 800 | <100 | <100 |
| C | Day 49 | Day 0; Day | 3200 | <100 | <100 |
| | | 21; Day 35 | 12800 | <100 | <100 |
| | | | 6400 | <100 | <100 |
| | | | 6400 | <100 | <100 |
| | | | 1600 | <100 | <100 |
| | | | 1600 | <100 | <100 |

As indicated in the final study report, the anti-gag and env ELISAs were developed using clade B HIV-1 antigens; cross reactivity with serum elicited against subtype C antigens is not known. Results from the env and gag ELISAs were inconclusive. None of the serum samples from the TBC-M4 vaccine (subtype C) immunized animals detected the subtype B gag antigen utilized in the manufacturer's ELISA assay. One of the 18 serum samples from the TBC-M4 vaccine (subtype C) immunized animals showed mild reactivity with the subtype B env antigen. The negative data with Clade B gag antigen was not expected given the reported conservation of gag antigenicity in Clade B and subtype C HIV-1 strains. However, results obtained with the env and gag ELISAs could not be interpreted since the ability of the current assay to detect antibody raised against subtype C antigens has not been established. Positive control serum with verified reactivity to subtype C env and/or gag antigen was not available.

The objective of this study was to verify biological activity of the vaccine in the CD1 mouse strain. The serum anti-vaccinia binding response was assayed in a validated ELISA method.

TBC-M4 vaccine was provided in a frozen state at 5×10⁸ pfu/ml. The test material and placebo (PBS/10% Glycerol) were stored frozen until use. On each dosing occasion the animals received 50 μl of undiluted, thawed test material or placebo delivered by intramuscular injection In alternating hind limbs. Animals were dosed and serum recovered. Blood was collected from each animal prior to SD 0 and at SD 78 two weeks following the fourth (final) dosing occasion. Pre- and post-immunization samples were assayed for serum vaccinia binding responses by ELISA. Reported titers were determined based on the OD value measured in native sera times three. The limit of detection is a titer of 100 which indicates that at a 1:100 dilution the OD of the sample is comparable to negative control walls.

Results of the anti-vaccinia binding ELISA are provided In Table 5. None of the serum samples collected prior to dosing, or samples from animals exposed to placebo, contained detectable anti-vaccinia titers. All of the serum samples from mice administered the TBC-M4 vaccine contained markedly elevated anti-vaccinia titers (range 25600 to 51200). A positive humoral response is indicated by a 2-fold increase of the anti-vaccinia titer in post-immunization over pre-dose titers. The serum titers of 25600 to 51200 in the vaccinated animals indicated a positive response to the vaccine and verified the pharmacologic activity of the vaccine in the CD1 mouse model utilized in a repeat dose toxicology study.

TABLE 5
Post-immunization antibody titers

| Group & Sex | Time point | Anti-Vaccinia titer |
|---|---|---|
| 4F | Pre-Dose | <100 |
| Placebo | SD 78 | <100 |
| | Pre-Dose | <100 |
| | SD 78 | <100 |
| | Pre-Dose | <100 |
| | SD 78 | <100 |
| | Pre-Dose | <100 |
| | SD 78 | <100 |
| | Pre-Dose | <100 |
| | SD 78 | <100 |
| 5F | Pre-Dose | <100 |
| TBC-M4 | SD 78 | 51200 |
| 2.5 × 10⁷ pfu/dose | Pre-Dose | <100 |
| | SD 78 | 51200 |
| | Pre-Dose | <100 |
| | SD 78 | 25600 |
| | Pre-Dose | <100 |
| | SD 78 | 51200 |
| | Pre-Dose | <100 |
| | SD 78 | 51200 |

Murine IFN.gamma ELISPOT. The objective of this study was to determine the cellular immune response of BALB/c mice to TBC-M4 vaccine by measuring the frequency of HIV1 antigen specific splenocytes in an IFN-gamma ELISPOT assay. This study is a proof-of-concept study conducted with peptide reagents synthesized for a related but not identical multigenic HIV-1 subtype C construct. The peptide pools were modeled and synthesized to include overlapping 15-mer amino acid sequences from env, gag, pol (RT) and nef-tat proteins.

TBC-M4 vaccine was provided in a frozen state at a stock concentration of 1×10⁹ pfu/ml. The test material was stored frozen until use. Animals were dosed on 1, 2, or 3 dosing occasions with test article at 1×10⁴ pfU 1×10⁶ pfU or 1×10⁸ pfu delivered per administration. On each day of test article administration new dosing solutions were prepared. Dose solution C (1×10⁸ pfu/0.1 m) required no preparation as the neat stock vaccine was provided at 1×10⁹ pfu/ml. Dose solution B (1×10⁶ pfu/0.1 ml) was prepared by making a two serial 1 to 10 dilutions of the stock vaccine in endotoxin free PBS. Dose solution A (1×10⁴ pfu/0.1 ml) was prepared by making two serial 1 to 10 dilutions Dose solution in endotoxin free PBS.

Female BALB/c mice were selected as the animal model based on previous experience with similar immunogenicity protocols. On each dosing occasion the animals received 100 μl of test material delivered in two 50 μl intramuscular injections, one into each of the two hind limbs. Animals were dosed and spleens recovered two weeks after each immunization.

Spleens were collected and transferred to the ELISPOT testing facility in complete media containing 2% fetal bovine serum. Spleens were received and processed for the ELISPOT assay on the same day of collection. Splenic lymphocytes were isolated and collected from each tissue sample using aseptic technique via tissue disaggregation. Single cell suspensions for each sample were counted and the concentrations adjusted to yield a final cell density of 2×10⁵ cells per well. Samples were tested in triplicate wells, for a total of 11 stimulation conditions including two controls: media alone (negative control) and Con A (a T-cell mitogen; positive control), and nine different peptide stimulations at 1.5-2 μg/μl. The HIV-1 peptide pools and single peptides utilized are described in Table 6. The cells and the stimulants were dispensed in 96-well ELISPOT filter-plates pre-coated with antimouse IFN-gamma antibody and incubated for 18-24 hours at 37° C. Remaining unused cells were frozen at −70° C. Enzyme labeled mouse IFN-gamma specific detector antibodies were used to detect the spots produced by the IFN-gamma secreted by the stimulated cells.

TABLE 6
Peptides used for ELISPOT assay

| | Peptide | Pool | | |
|---|---|---|---|---|
| Gene | Numbers | Composition | Pool name | Origin |
| gag | HIVC.2 | Peptide 1-119 | HIVC.2-p1 | Overlapping 15- |
| | (1-119) | | | mer peptide |
| pol | HIVC.4 | Peptide 1-116 | HIVC.4-p2 | pools were |
| | (1-233) | Peptide 117-233 | HIVC.4-p3 | derived from |
| env | HIVC.5 | Peptide 1-101 | HIVC.5-p4 | subtype C HIV |
| | (1-202) | Peptide 101-202 | HIVC.5-p5 | gene sequences |
| nef-tat | HIVC.6 | Peptide 1-74 | HIVC.6-p5 | similar to those |
| | | | | encoded by the |
| | | | | TBC-M4 |
| | | | | vaccine. Rev |
| | | | | peptides were not |
| | | | | available |
| | Peptide | Pool | | |
| Mouse epitopes | Name | Composition | Full Peptide Sequence | Origin |
| Env: | HIVC.5-28 | Not | SNGTYNETYNEIKNCS | Experimentally |
| TYNETYNEI | | applicable; | | derived single |
| Gag: | HIVA.16 | Not pools | HQAAMQMLKDTINEE | peptides known |
| AMQMLKDTI | | | | to be biologically |
| Pol: | HIVC.4-103 | | VHGAYVOPSKDLIAE | active with H-2D |
| YYDPSKDLI | | | | splenocytes from |
| | | | | animals exposed |
| | | | | to subtype C |
| | | | | gene sequences |
| | | | | similar to those |
| | | | | encoded by the |
| | | | | TBC-M4 vaccine |

A summary of the results from the ELISPOT assay is provided in Table 7. EliSPOT results are reported as the number of IFN-gamma producing cells per well (2×10⁵ cells). Values greater than or equal to the mean value in the negative control (unstimulated wells) plus 2 SD are considered positive in the assay.

Env, gag and pol (RT) specific responses were observed in cell cultures from all animals immunized with TBC-M4 at all doses tested (1×10⁴ to 1×10⁸ pfu). HIV-1 antigen (peptide) specific responses were not detected in spleen cell cultures from naive animals. Since in vitro stimulation of naive cells failed to induce detectable vaccine specific IFN-gamma producing cells, the observed responses were attributed to the in vivo stimulation of host immune cells by the TBC-M4 vaccine.

TABLE 7
IFN-gamma ELISPOT raw data

| In vivo | | | | | | | | | | | |
|---|---|---|---|---|---|---|---|---|---|---|---|
| TBC-M4 | In vitro stimulation | | | | | | | | | | |
| Dose | | | gag | pol | pol | env | env | nef/tat | env | gag | pol |
| (pfu) | None | Con A | 1-119 | 1-116 | 117-233 | 1-101 | 101-202 | 1-74 | pept | pept | pept |
| | Post First Immunization (SD 14) | | | | | | | | | | |
| 1 × 10⁴ | 1 | 638 | 22 | 8 | 1 | 17 | 9 | 1 | 0 | 2 | 1 |
| | 1 | 583 | 16 | 6 | 0 | 21 | 4 | 2 | 1 | 6 | 1 |
| | 0 | 630 | 15 | 8 | 2 | 18 | 11 | 0 | 1 | 1 | 3 |
| | 1 | 602 | 29 | 17 | 1 | 19 | 3 | 2 | 1 | 0 | 2 |
| 1 × 10⁶ | 1 | 666 | 12 | 12 | 2 | 36 | 7 | 3 | 1 | 1 | 1 |
| | 0 | 679 | 19 | 9 | 2 | 29 | 8 | 1 | 2 | 2 | 2 |
| | 1 | 704 | 10 | 7 | 1 | 18 | 7 | 2 | 1 | 1 | 1 |
| | 0 | 686 | 25 | 10 | 1 | 31 | 12 | 2 | 0 | 7 | 1 |
| | 1 | 565 | 11 | 6 | 0 | 11 | 4 | 0 | 0 | 2 | 0 |
| 1 × 10¹⁰ | 1 | 597 | 24 | 13 | 3 | 72 | 22 | 2 | 2 | 5 | 2 |
| | 0 | 664 | 16 | 16 | 2 | 37 | 14 | 2 | 1 | 3 | 7 |
| | 1 | 626 | 7 | 4 | 0 | 24 | 5 | 3 | 0 | 1 | 1 |
| | 3 | 657 | 49 | 15 | 4 | 58 | 18 | 4 | 3 | 10 | 6 |
| | 4 | 659 | 40 | 37 | 8 | 85 | 10 | 5 | 3 | 8 | 11 |
| None | 0 | 664 | 0 | 0 | 1 | 0 | 0 | 2 | 1 | 1 | 1 |
| | 0 | 644 | 0 | 1 | 0 | 0 | 0 | 0 | 0 | 0 | 0 |
| | Post Second Immunization (SD 35) | | | | | | | | | | |
| 1 × 10⁴ | 0 | 585 | 23 | 9 | 1 | 19 | 8 | 0 | 0 | 2 | 0 |
| | 0 | 625 | 24 | 7 | 0 | 23 | 18 | 0 | 0 | 1 | 0 |
| | 0 | 599 | 14 | 9 | 0 | 8 | 4 | 0 | 0 | 1 | 1 |
| | 0 | 693 | 14 | 10 | 1 | 18 | 5 | 0 | 0 | 1 | 1 |
| | 0 | 660 | 19 | 15 | 0 | 20 | 4 | 0 | 0 | 14 | 0 |
| 1 × 10⁶ | 1 | 492 | 26 | 12 | 0 | 54 | 17 | 1 | 0 | 13 | 1 |
| | 3 | 474 | 54 | 13 | 1 | 40 | 18 | 0 | 1 | 3 | 1 |
| | 1 | 881 | 43 | 33 | 1 | 63 | 18 | 1 | 0 | 25 | 8 |
| | 0 | 604 | 27 | 19 | 1 | 40 | 9 | 1 | 1 | 7 | 5 |
| | 1 | 695 | 15 | 23 | 0 | 37 | 27 | 1 | 0 | 3 | 3 |
| 1 × 10¹⁰ | 4 | 337 | 48 | 55 | 5 | 241 | 19 | 3 | 1 | 12 | 9 |
| | 2 | 500 | 29 | 109 | 3 | 139 | 15 | 4 | 1 | 46 | 26 |
| | 4 | 558 | 100 | 91 | 5 | 224 | 10 | 5 | 4 | 5 | 23 |
| | 2 | 430 | 59 | 171 | 2 | 227 | 16 | 1 | 1 | 7 | 38 |
| | 1 | 699 | 28 | 17 | 3 | 53 | 20 | 4 | 1 | 3 | 4 |
| None | 0 | 637 | 1 | 0 | 0 | 0 | 0 | 0 | 0 | 0 | 0 |
| | 0 | 801 | 0 | 0 | 0 | 0 | 0 | 1 | 0 | 0 | 0 |
| | Post Third Immunization (SD 49) | | | | | | | | | | |
| 1 × 10⁴ | 0 | 869 | 24 | 12 | 0 | 16 | 8 | 1 | 0 | 17 | 6 |
| | 0 | 905 | 21 | 13 | 2 | 23 | 5 | 0 | 0 | 0 | 1 |
| | 1 | 916 | 10 | 11 | 1 | 11 | 2 | 0 | 0 | 3 | 1 |
| | 0 | 866 | 40 | 16 | 1 | 40 | 13 | 0 | 0 | 3 | 0 |
| | 0 | 972 | 42 | 8 | 0 | 23 | 6 | 0 | 0 | 2 | 1 |
| 1 × 10⁶ | 1 | 1084 | 38 | 14 | 2 | 41 | 6 | 4 | 0 | 5 | 5 |
| | 2 | TNTC | 60 | 56 | 2 | 63 | 30 | 1 | 2 | 17 | 16 |
| | 1 | TNTC | 44 | 93 | 5 | 83 | 14 | 1 | 1 | 32 | 16 |
| | 0 | 324 | 50 | 51 | 2 | 35 | 11 | 0 | 1 | 16 | 12 |
| | 1 | TNTC | 21 | 41 | 2 | 59 | 15 | 2 | 0 | 10 | 15 |
| 1 × 10¹⁰ | 2 | 915 | 10 | 24 | 2 | 36 | 4 | 1 | 2 | 4 | 7 |
| | 3 | 880 | 18 | 22 | 1 | 76 | 7 | 3 | 1 | 2 | 4 |
| | 4 | 417 | 57 | 114 | 4 | 146 | 24 | 3 | 2 | 37 | 29 |
| | 6 | 1079 | 47 | 37 | 6 | 187 | 30 | 3 | 4 | 56 | 12 |
| | 1 | 974 | 12 | 52 | 3 | 26 | 5 | 0 | 1 | 3 | 12 |
| None | 1 | 849 | 0 | 0 | 0 | 0 | 0 | 0 | 1 | 0 | 0 |
| | 1 | 854 | 0 | 0 | 0 | 0 | 1 | 1 | 0 | 0 | 0 |

TNTC—Too numerous to count

The magnitude of responses to env, gag and pol (RT) roughly correlated with the dose of vaccine administered indicating a dose dependent immune response to the vaccine. IFN gamma responses were detected in all three-dosage groups following the primary exposure to vaccine. The magnitude of responses was generally higher following each subsequent administration of vaccine. However, splenocytes from animals receiving 3 exposures to the highest dosage (1×10⁸ pfu) appeared refractory to stimulation when compared to splenocytes from the same dosage group receiving two exposures.

Similar patterns of antigen specific IFN-gamma stimulation were observed in cultures stimulated with single peptides from gag and pol (RT) but the responses were less consistent between animals and were of a lower magnitude. The single env peptide epitope was not stimulatory (data not shown) nor was the peptide pool derived from a similar subtype C nef-tat fusion polypeptide (data not shown). The lack of response to a single env peptide is not unexpected, when tested to multiple peptides contained in the env pools (env (1) and env (2)) a response was revealed to this encoded HIV protein. Subsequent analysis of the nef peptide pool used for re-stimulation revealed a match of only 9 of 51 epitope sequences between the nef peptide pool utilized and a theoretical TBC-M4 vaccine matched nef pool. The observed differences between the two nef polypeptide pools suggests that the poor responses to nef (and tat) were related to the lack of suitable reagents.

Thus, responses were detected to three of six target HIV-1 antigen inserts and suitable reagents were not available for measurement of the response to the remaining three. The IFN-gamma response following TBC-M4 administration, is considered indicative of T cell stimulatory activity of the vaccine. The pharmacologic effect of T8C-M4 is affected by the number of administrations and the amount of vaccine administered on each dosing occasion.

Murine IFN-gamma ELISPOT. The objective of this study was to determine the immune response to the TBC-M4 vaccine in BALB/c and CD1 murine splenocytes by IFN-gamma ELISPOT assay. This study was a proof-of-concept study conducted with env, gag and pol (RT) peptide reagents synthesized for a related but not identical multigenic HIV-1 subtype C construct. Peptide pools shown to be active in the above study were utilized during the in vitro stimulation phase of the IFN-gamma ELISPOT assay. The peptide pools were modeled and synthesized to include overlapping 15-mer amino acid sequences from env, gag, pol (RT) and nef-tat proteins.

TBC-M4 vaccine was provided in a frozen at a stock concentration of 5×10⁸ pfu/ml. The test material was stored frozen until use. Female BALB/c mice were selected as the animal model based on previous experience with similar immunogenicity protocols. CD1 mice were selected to verify pharmacologic activity of the vaccine in this mouse strain. On each dosing occasion the animals received 100 μl of undiluted, test material delivered in two 50 μl intramuscular injections, one into each of the two hind limbs. Animals were dosed and spleens recovered two weeks after the second administration (SD 35). Blood was collected at the time of spleen harvest. Serum was stored frozen.

Spleens were collected and transferred to the ELISPOT testing facility in complete media containing 2% fetal bovine serum. Spleens were processed for the ELISPOT assay on the same day of collection. Splenic lymphocytes were isolated and collected from each tissue sample using aseptic technique via tissue disaggregation. Single cell suspensions for each sample were counted and the concentrations adjusted to yield a final cell density of 2×10⁵ cells per well. Samples were tested in triplicate wells, for a total of 7 stimulation conditions including two controls: media alone (negative control) and Con A (a T-ell mitogen; positive control), and five different HIV-1 peptide pools at 1.5-2 μg/ml. The HIV-1 peptide pools are described in Table 8. The cells and the stimulants were dispensed in 96 well ELISPOT filter-plates pre-coated with anti-mouse IFN-gamma antibody and incubated for 18-24 hours at 37° C. Remaining, unused cells were frozen at −70° C. Enzyme labeled mouse IFN-gamma specific detector antibodies were used to detect the spots produced by the IFN-gamma secreted by the stimulated cells.

TABLE 8
Peptides used for ELISPOT Assay

| | Peptide | Pool | | |
|---|---|---|---|---|
| Gene | Numbers | Composition | Pool name | Origin |
| gag | HIVC.2 | Peptide 1-119 | HIVC.2-p1 | Overlapping 15-mer |
| | (1-119) | | | peptide pools were |
| pol | HIVC.4 | Peptide 1-116 | HIVC.4-p2 | derived from |
| | (1-233) | Peptide 117-233 | HIVC.4-p3 | subtype C HIV |
| env | HIVC.5 | Peptide 1-101 | HIVC.5-p4 | gene sequences |
| | (1-202) | Peptide 101-202 | HIVC.5-p5 | similar to those |
| | | | | encoded by the |
| | | | | TBC-M4 vaccine. |

The filter ELISPOT plates were scanned on a CT1 Immunospot Scanner for spot-pictures of the 96-wells. The CTL Immunospot analyzer software was used to count the number of spots in each well. The mean of triplicate values was derived using excel template with inbuilt formulae.

A summary of the results from the ELISPOT assay is provided in Table 9. ELISPOT results are reported as the number of IFN-gamma producing cells per well (2×10⁵ cells). Values greater than or equal to the mean value In the negative control (unstimulated wells) plus 2 SD are considered positive in the assay.

TABLE 9
IFN-gamma ELISPOT raw data

| Mouse | | | | | | | |
|---|---|---|---|---|---|---|---|
| Strain | | | | | | | |
| In vivo | In vitro stimulation | | | | | | |
| TBC-M4 | | | gag | pol | pol | env | env |
| Dose | None | ConA | 1-119 | 1-116 | 117-233 | 1-101 | 101-202 |
| BALB/c | 6 | TNTC | 148 | 436 | 17 | 187 | 45 |
| 5 × 10⁷ | 21 | TNTC | 97 | 126 | 18 | 302 | 83 |
| pfu | 41 | TNTC | 138 | 213 | 26 | 384 | 75 |
| | 21 | TNTC | 312 | 337 | 34 | 389 | 165 |
| | 24 | TNTC | 58 | 135 | 13 | 300 | 48 |
| None | 3 | 786 | 1 | 0 | 1 | 2 | 2 |
| | 2 | TNTC | 3 | 3 | 1 | 3 | 4 |
| CD1 | 22 | TNTC | 96 | 33 | 18 | 364 | 304 |
| 5 × 10⁷ | 27 | TNTC | 67 | 64 | 23 | 320 | 115 |
| pfu | 13 | TNTC | 100 | 31 | 18 | 480 | 558 |
| | 29 | TNTC | 49 | 46 | 42 | 24 | 674 |
| | 24 | TNTC | 123 | 57 | 33 | 507 | 459 |
| | 8 | TNTC | 69 | 42 | 25 | 331 | 50 |
| | 39 | TNTC | 92 | 56 | 47 | 464 | 254 |
| | 51 | TNTC | 200 | 117 | 69 | 432 | 171 |
| | 16 | TNTC | 78 | 51 | 22 | 232 | 676 |
| | 15 | TNTC | 52 | 40 | 15 | 364 | 125 |
| None | 0 | 792 | 2 | 4 | 1 | 6 | 5 |
| | 24 | 800 | 114 | 132 | 63 | 95 | 46 |

TNTC—Too numerous to count

Cell cultures from animals immunized with TBC-M4 vaccine demonstrated IFN-gamma responses following in vitro stimulation with the env, gag or pol (RT) peptide pools. The magnitude and pattern of the T-cell associated IFN-gamma responses In the BALB/c mice verified the positive results reported previously. The magnitude of the T-cell responses to the gag and env components was comparable in CD1 and BALB/c mice. Pol (RT) associated response appeared stronger in splenocytes from BALB/c mice. Antigen (peptide) specific responses were not detected in BALB/c spleen cell cultures from naive animals.

Unexpectedly the cell cultures from one of two naive CD1 mice responded to the HIV-1 peptide pools. Review of the assay and the responses indicated that the spot pattern in that animal number was distinct from that in the immunized animals with a higher than expected variation among triplicate wells and qualitative differences noted by the operator. The factors contributing to the unexpected response were investigated but no single assignable cause could be determined. Potential factors that may have contributed to the unexpected result include operator error during immunization and/or assay conduct or the outbred background of the CD1 mice. The conclusions of the investigation are as follows:
The test article induced a robust cellular immune response in 100% of the vaccinated mice.
The issue of an apparent response in one of the two CD1 negative control mice is most plausibly explained by a background response to the HIV peptides in this animal, although other causes [operator errors, etc] cannot be ruled out completely.
The CD1 background explanation is supported by the absence of response in two negative control BALB/c mice, and the magnitude of response in the negative control mouse, plus investigation into assay conduct.

In summary, the observed antigen specific IFN-gamma response to three of the six target HIV proteins (env, gag and RT) in these studies verify the intended effect of the vaccine, namely induction of immune responses to the HIV proteins encoded by the TBC-M4 product. The IFN-gamma response following TBC-M4 administration, is considered indicative of T-cell responsiveness to the HIV antigen components of the vaccine. These results re-affirm the pharmacologic activity of the TBC-M4 vaccine in animals exposed by the intramuscular route of administration.

Immunogenicity of TBC-M4 in rabbits. The objective of this study was to verify biological activity of the vaccine in the New Zealand White (NZW) rabbit animal model. Serum collected from NZW rabbits, pre- and post-vaccination, was tested for the presence of vaccinia binding antibodies using a qualified ELISA method.

TBC-M4 vaccine was provided in a frozen state: clinical Lot 1A (5×10⁸ pfu/ml) and clinical Lot 1B (1×10⁸ pfulml). The test material and placebo (PBS/10% Glycerol) were stored frozen until use. Clinical lot 1A. Clinical lot 1B and placebo were used to dose animals in alternating limb regions as specified in the protocol: SD1 (left). SD 22 (right), SD 43 (left), and SD 64 (right). On each dosing occasion the animals received 0.5 μl of undiluted, thawed test material or placebo delivered by intramuscular injection in the hind limb alternating left/right as above. Animals were dosed and serum recovered.

Blood (1 ml) was collected from each animal prior to any test article administration (prestudy) and again at SD 67, three days following the fourth (final) dosing occasion. Pre- and post-vaccination blood was collected from the ear vein or artery. Serum was collected following standard clotting and centrifugation procedures. Pre- and post-immunization samples were collected for assay of vaccinia binding antibody responses by ELISA.

Titers were determined based on the value of the naive sera times three. A positive response was indicated by a 2-fold increase of the post-immunization samples when compared to Pre-dose sample.

Results of the anti-vaccinia binding ELISA are provided in Table 10. Prior to test article administration, the serum anti-vaccinia titers were at the limit of detection (≦100) in 34 of the 36 study animals. Two animals had serum titers of 400 at the initiation of the study, which may indicate previous exposure to vaccinia cross-reactive antigens in a small subset of animals.

TABLE 10
Post-immunization antibody titers

| | PreDose | SD67 | | PreDose | SD67 |
|---|---|---|---|---|---|
| Group/Sex | Anti-vaccinia titer | | Group/Sex | Anti-vaccinia titer | |
| 1M | <100 | <100 | 1F | <100 | <100 |
| | <100 | <100 | | <100 | <100 |
| | <100 | <100 | | 400 | 400 |
| | <100 | <100 | | <100 | <100 |
| | <100 | <100 | | 100 | 100 |
| | 100 | <100 | | 100 | 100 |
| 2M | 100 | 25600 | 1F | <100 | 25600 |
| | 100 | 25600 | | <100 | 25600 |
| | 100 | 25600 | | <100 | 25600 |
| | <100 | 25600 | | <100 | 25600 |
| | 100 | 25600 | | <100 | 25600 |
| | <100 | 25600 | | 400 | 6400 |
| 3M | <100 | 25600 | 1F | <100 | 102400 |
| | <100 | 10200 | | <100 | 25600 |
| | 100 | 25600 | | <100 | 25600 |
| | <100 | 25600 | | <100 | 25600 |
| | <100 | 25600 | | <100 | 102400 |
| | 100 | 25600 | | <100 | 25600 |

None of the 12 rabbits in Group 1, control group, showed a positive binding response to the vaccinia antigen in the ELISA, i.e. no increase in antibody titer in SD 67 sera as compared to titers in pre-dose sera. All 24 rabbits that received the TBC-M4 vaccine (Groups 2: 5×10⁷ pfu and Group 3: 2.5×10⁸ pfu) seroconverted to vaccinia. Titers from group 2 (low dose) animals ranged from 6400 to 25600. Titers from group 3 (high dose) animals ranged from 25600 to 102400. The positive seroconversion of all animals receiving TBC-M4 verified the pharmacologic activity of the vaccine in the rabbit model selected for toxicological testing.

In summary, five in vivo studies were conducted to assess the biologic activity of the TBC-M4 vaccine in animals; four were conducted in mice and one was conducted in rabbits. The pharmacologic activity of the vaccine, including the attenuated vaccinia vector and the inserted HIV gene products, was demonstrated by elicitation of host immune responses to multiple vaccine components. Humoral responses to the vaccine vector were observed in three of three studies. Responses to the inserted HIV-1 gene products were observed in the two proof-of-principle studies. In both studies significant IFN-gamma responses were observed to the env, gag, and pol (RT) antigens. Together the results of these studies support the phase I clinical testing of the TBC˜M4 vaccine candidate.

### Example 4

### Animal Toxicology

Two repeat dose non-clinical safety studies were conducted with the proposed clinical lots of TBC-M4 vaccine. Both mice and rabbits were exposed to 4 intramuscular injections of placebo or TBC-M4, at three week intervals, in alternating hind limbs. Four repeated injections were delivered to represent the proposed clinical regimen (3 dosages) plus one. Dose level selection was conducted to deliver the maximal allowable volume in mice and to deliver a full human dose equivalent to rabbits.

The doses for murine study were selected to deliver the maximum volume of test article that can be delivered In this species using 50 μl of the two proposed clinical doses; Lot A at 5×10⁸ pfu/mL and Lot B at 1×10⁸ pfu/mL. The 50 μl volume corresponds to an approximate 1/10 of the full human dosage proposed for delivery to humans in 0.5 mL. For Lot 1B, the dose delivered on a dose/kg basis represents a 50 fold increase over the highest clinical dose for humans; assuming an average human weighs 70 kilogram (kg) and a-week old mouse 30 grams. For Lot 1A, the dose delivered on a dose/kg basis represents a 230 fold increase over the highest clinical dose intended for humans.

The doses delivered in the rabbit study represents a full human dose of the highest proposed clinical dose (Lot 1A: 5×10⁸ pfu/mL) and a second sublot, clinical Lot 1A, filled at 1×10⁸ pfu/mL. On a dose per/kg basis this represents a minimum of a 20-fold safety margin over the highest clinical dose intended for humans; assuming an average human weighs 70 kilograms (kg) and a young adult rabbit weights 3.5 kilograms (kg).

The two non-clinical safety studies were conducted independently. Both studies included monitoring of mortality, clinical and cageside observations. body weights, body weight changes, food consumption, ophthalmology, necropsy, organ weights and ratios, and clinical pathology parameters (hematology and clinical chemistry). Microscopic analysis of a standard tissue battery was conducted In mice and for the injection sites in rabbits.

Repeat intramuscular injection of placebo (PBS/glycerol) or the TBC-M4 vaccine was well tolerated in both the rabbit and mouse animal models. Test article related observations in both animal models included a reversible mild to moderate local reactivity at the site of injection that was apparent both macroscopically as measured by draize scoring and microscopically in histopathology of biopsies from the injection sites. Other test article related changes in the mouse study included higher globulin levels, lymph node enlargement, and splenic white pulp hyperplasia which were considered attributable to the intended immune response to the vaccine. In the rabbit study there was a higher incidence of red skin and/or scabbing in the neck region of the treated females. In the absence of a dose response or similar observation in the males, the change was considered incidental, although a relation to the treatment could not be ruled out.

### Example 5

### Comparison of Vector Based HIV Vaccines Immunogenicity: ELISPOT-IFN-Gamma

Table 11 provides a comparison of vector Based HIV Vaccines immunogenicity based upon ELISPOT-IFN-gamma. Vaccine response rate in vaccines at peak post vaccination time-point per trial; Core Laboratory generated data; GMT SFC and min max SFC for responders; background subtracted per 106 PBMCs.

TABLE 11

| | | | | | | | |
|---|---|---|---|---|---|---|---|
| DNA | DNA | DNA | AAV | MVA | MVA | MVA | Adeno |
| “O” | “A” | “V” | “T” | “O” | “A” | TBC-M4 | “V” |
| 2 mg | 3 × 4 mg | 3 × 4 mg | 1 × 10¹¹ | 5 × 10⁷ | 2.5 × 10⁸ | 2.5 × 10⁸ | 1 × 10¹⁰ |

Responders

| | | | | | | | |
|---|---|---|---|---|---|---|---|
| 6% | 17% | 49% | 20% | 5% | 62% | 92% | 46% |

Geometric Mean: SFC/million and Range of Responses

| | | | | | | | |
|---|---|---|---|---|---|---|---|
| 35 | 69 | 109 | 130 | 57 | 130 | 80 | 101 |
| 31-40 | 66-73 | 44-598 | 54-385 | 41-79 | 55-275 | 39-193 | 52-297 |

The invention may be further described by the following numbered paragraphs:

1. A method for obtaining an immunogenic response against HIV-1 comprising administering to a mammal: an immunological composition against one or more immunogens comprising a MVA containing and expressing a nucleotide sequence encoding one or more HIV-1 immunogens.

2. A method for obtaining an immunogenic response against HIV-1 comprising administering to a mammal: (a) an immunological composition against a first immunogen comprising a MVA containing and expressing a nucleotide sequence encoding one or more HIV-1 immunogens; and (b) an immunological composition against one or more HIV-1 immunogens comprising a MVA containing and expressing a nucleotide sequence encoding the second immunogen of a pathogen of the mammal, wherein (a) and (b) are administered sequentially.

3. The method of paragraph 2 wherein (a) and (b) are sequentially administered, whereby there is a first administration of (a), followed by a subsequent administration of (b).

4. The method of paragraph 2 wherein (a) and (b) are sequentially administered, whereby there is a first administration of (b), followed by a subsequent administration of (a).

5. The method according to any one of paragraphs 2-4 wherein the first immunogen and the second immunogen are the same immunogen.

6. The method of any one of paragraphs 2-5 wherein a prime boost regimen is used.

7. The method of any one of paragraphs 1-6 wherein the mammal is a human.

8. The method of any one of paragraphs 1-7 wherein the HIV-1 immunogens are selected from the group consisting of HIV proteins encoded by the env, gag, nef, reverse transcriptase (RT), tat and rev genes, or a fragment thereof.

9. The method of any one of paragraphs 1-8 wherein the HIV-1 immunogens are encoded by the TBC-M4 HIV gene sequence insert.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method for eliciting an immunogenic response against HIV-1 comprising administering to a mammal: an immunological composition against one or more immunogens comprising a MVA containing and expressing a nucleotide sequence encoding one or more HIV-1 immunogens, wherein the HIV-1 immunogens are selected from the group consisting of HIV proteins encoded by the env, gag, nef, reverse transcriptase (RT), tat and rev genes or a fragment thereof.

2. The method of claim 1, wherein the HIV-1 immunogens are HIV-1 subtype C immunogens.

3. The method of claim 1, wherein the HIV-1 immunogen comprises a full-length env.

4. The method of claim 3, wherein the full-length env is modified to introduce silent mutations to internal motifs that encode early transcription termination signals.

5. The method of claim 1, wherein the HIV-1 immunogen comprises a full-length gag.

6. The method of claim 1, wherein the HIV-1 immunogen comprises a tat and rev fusion gene.

7. The method of claim 1, wherein the HIV-1 immunogen comprises a modified reverse transcriptase (RT) portion of the pol gene, wherein the modification eliminates reverse transcriptase activity.

8. The method of claim 1, wherein the HIV-1 immunogen comprises a nef-RT fusion gene.

9. The method of claim 1, wherein the HIV-1 immunogens comprise a full-length env, a full-length gag, a tat and rev fusion gene, a modified reverse transcriptase (RT) portion of the pol gene, wherein the modification eliminates reverse transcriptase activity and a nef-RT fusion gene.

10. The method of claim 1, wherein the mammal is a human.
